(19) United States
(12) Patent Application Publication
(10) Pub. No.: US 2016/0263184 A1
(43) Pub. Date: Sep. 15, 2016

(54) METHODS OF CONTROLLING VASCULARITY USING RAVER2 AS A MEDIATOR FOR EXPRESSION OF VEGF RECEPTOR SFIT1

(71) Applicant: UNIVERSITY OF UTAH RESEARCH FOUNDATION, Salt Lake City, UT (US)

(72) Inventors: Balamurali K. AMBATI, Sandy, UT (US); Derick G. HOLT, South Jordan, UT (US)

(21) Appl. No.: 14/765,226

(22) PCT Filed: Feb. 3, 2014
(86) PCT No.: PCT/US2014/014498

§ 371 Date: Jul. 31, 2015

Related U.S. Application Data
(60) Provisional application No. 61/759,947, filed on Feb. 1, 2013.

(51) Int. Cl. A61K 38/17; C12N 15/113; A61K 9/00

(52) U.S. Cl. CPC A61K 38/1709; A61K 9/0048; C12N 15/113; C12N 2310/122; C12N 2310/14; C12N 2310/531; C12N 2320/32

(57) ABSTRACT

Methods of treating a condition in a subject resulting from abnormally high VEGF signaling through membrane-bound receptors. Such a method can include increasing expression of Raver2 in affected cells of the subject to increase production of soluble VEGF receptors. In some aspects, increasing expression of Raver2 decreases production of membrane-bound VEGF receptors.

### GOVERNMENT INTEREST

This invention was made with government support under grant No. NEI 5R01EY017950 from the National Institute of Health. The United States government has certain rights to this invention.

### BACKGROUND

Vascular compartmentalization in the eye is striking in the cornea, which must remain clear for optimal vision. The clarity of the cornea is endangered by adjacent episcleral and conjunctival blood vessels that can invade it in multiple pathologic conditions, leading to conical neovascularization (KNV) with resultant opacification and vision loss. This is a condition affecting millions of individuals, as corneal blindness currently represents the second leading cause of vision loss worldwide.

The cornea's physiologic vascular zoning ability derives from soluble vascular endothelial growth factor receptor -1 (sVEGFR-1, also known as sFlt1) (Ambati et al. Nature 2006; 443:993-7). The biological scope of sFlt-1 now extends to normal vascular development, peripartum cardiomyopathy, preeclampsia, congenital vascular malformations, and cancer. sFlt1 is an alternate isoform of membrane-bound. VEGF receptor-1 (mVEGFR-1, also known as mFlt1), generated via an uncommon and poorly understood form of alternative RNA processing, intronic cleavage and polyadenylation (C/P). Despite its broad relevance, the molecular factor(s) that regulate sFlt1 production remain elusive.

### SUMMARY OF THE INVENTION

The present disclosure provides methods and compositions, including biotechnological compositions, for the treatment of conditions resulting from abnormal levels of angiogenesis. In one aspect, for example, a method of treating a condition resulting from abnormally high VEGF signaling through membrane-bound VEGF receptors can include administering to a subject in need of such treatment an effective amount of a polypeptide having a sequence region with at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof, wherein the polypeptide upregulates soluble VEGF receptor production in affected cells to decrease the abnormally high VEGF signaling through the membrane-bound VEGF receptors. In another aspect, the sequence region has 100% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof. In another aspect, the polypeptide is Raver2. In yet another aspect, the polypeptide has at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, or SEQ ID 011, wherein the polypeptide upregulates soluble VEGF receptor production in affected cells to decrease the abnormally high VEGF signaling through the membrane-bound VEGF receptors. In a further aspect, the polypeptide has 100% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, or SEQ ID 011.

In other aspects, administering an effective amount of the polypeptide further includes administering an effective amount of a polynucleotide encoding the polypeptide or potypeptide region, wherein the polynucleotide has at least 85% sequence identity to SEQ ID 012. In another aspect, the polynucleotide has at least 90% sequence identity to SEQ ID 012. In yet another aspect, the polynucleotide has at least 95% sequence identity to SEQ ID 012. In a further aspect, the polynucleotide has 100% sequence identity to SEQ ID 012.

A variety of conditions are contemplated that can be treated or otherwise ameliorated by methods according to aspects of the present disclosure. Non-limiting examples can include cancer, macular degeneration, diabetic retinopathy, rheumatoid arthritis, corneal injury, corneal transplant rejection, and the like, including appropriate combinations thereof.

The present disclosure additionally provides methods of treating a condition in a subject resulting from abnormally high VEGF signaling through membrane-bound receptors. Such a method can include increasing expression of Raver2 in affected cells of the subject to increase production of soluble VEGF receptors. In some aspects, increasing expression of Raver2 decreases production of membrane-bound VEGF receptors.

The present disclosure additionally provides methods of treating a condition in a subject resulting from abnormally low VEGF signaling through membrane-bound receptors. Such a method can include decreasing expression of Raver2 in affected cells of the subject to decrease production of soluble VEGF. In some aspects, decreasing expression of Raver2 increases production of membrane-bound VEGF receptors. A variety of conditions are contemplated that can be treated or otherwise ameliorated by methods according to aspects of the present disclosure. Non-limiting examples can include preeclampsia, heart disease, wound heating, stroke, and the like, including appropriate combinations thereof.

The present disclosure additionally provides pharmaceutical compositions for treating a condition or conditions resulting from abnormally high VEGF signaling through membrane-bound VEGF receptors. Such a composition can include at least one of 1) an effective amount of a polypeptide having a sequence region with at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof, or 2) an effective amount of a polynucleotide encoding a polypeptide having a sequence region with at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof and having a polynucleotide sequence that has at least 85% sequence identity to SEQ ID 012, and a pharmaceutically acceptable carrier. In one specific aspect, the composition is formulated as an ocular pharmaceutical composition.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### BRIEF DESCRIPTION OF THE DRAWINGS

For a fuller understanding of the nature and advantage of the present disclosure, reference is being made to the following detailed description of various embodiments and in connection with the accompanying drawings, in which:

FIG. 1A shows heat map data in accordance with an aspect of the present disclosure.

FIG. 1B shows graphical data in accordance with another aspect of the present disclosure.

FIG. 2 shows clustered data according to mouse strain in accordance with another aspect of the present disclosure.

FIG. 3 shows heat map data in accordance with another aspect of the present disclosure.

FIG. 4 shows graphical data in accordance with another aspect of the present disclosure.

FIG. 5A shows graphical data in accordance with another aspect of the present disclosure.

FIG. 5B provides an image of a gel showing data in accordance with another aspect of the present disclosure.

FIG. 5C shows graphical data in accordance with another aspect of the present disclosure.

FIG. 5D shows graphical data in accordance with another aspect of the present disclosure.

FIG. 6A provides an image of a gel showing data in accordance with another aspect of the present disclosure.

FIG. 6B provides an image of a gel showing data in accordance with another aspect of the present disclosure.

FIG. 7A provides an image of a gel showing data in accordance with another aspect of the present disclosure.

FIG. 7B provides an image of a gel showing data in accordance with another aspect of the present disclosure.

FIG. 7C shows an image of immunological staining in accordance with another aspect of the present disclosure.

FIG. 8A provides an image of a gel showing data in accordance with another aspect of the present disclosure.

FIG. 8B provides an image of a gel showing data in accordance with another aspect of the present disclosure.

FIG. 8C provides an image of a gel showing data in accordance with another aspect of the present disclosure.

FIG. 9A provides an image of a gel showing data in accordance with another aspect of the present disclosure.

FIG. 9B shows graphical data in accordance with another aspect of the present disclosure.

FIG. 10A shows graphical data in accordance with another aspect of the present disclosure.

FIG. 10B shows graphical data in accordance with another aspect of the present disclosure.

FIG. 11A provides an image of a gel showing data in accordance with another aspect of the present disclosure.

FIG. 11B shows graphical data in accordance with another aspect of the present disclosure.

FIG. 11C shows graphical data in accordance with another aspect of the present disclosure.

FIG. 11D shows graphical data in accordance with another aspect of the present disclosure.

FIG. 11E shows graphical data in accordance with another aspect of the present disclosure.

FIG. 11F shows graphical data in accordance with another aspect of the present disclosure.

FIG. 11G provides an image of a gel showing data in accordance with another aspect of the present disclosure.

FIG. 11H shows graphical data in accordance with another aspect of the present disclosure.

FIG. 12A shows graphical data in accordance with another aspect of the present disclosure.

FIG. 12B shows images of mouse corneas post injection in accordance with another aspect of the present disclosure.

FIG. 12B shows images of mouse corneas in accordance with another aspect of the present disclosure.

FIG. 12C shows images of mouse corneas in accordance with another aspect of the present disclosure.

FIG. 12D shows graphical data in accordance with another aspect of the present disclosure.

FIG. 12E shows graphical data in accordance with another aspect of the present disclosure.

FIG. 12F shows images of mouse corneas in accordance with another aspect of the present disclosure.

FIG. 12G shows graphical data in accordance with another aspect of the present disclosure.

FIG. 13A shows an image of human corneal epithelium in accordance with another aspect of the present disclosure.

FIG. 13B shows an image of human corneal epithelium accordance with another aspect of the present disclosure.

FIG. 13C shows an image of human corneal epithelium in accordance with another aspect of the present disclosure.

FIG. 13D shows an image of human conical epithelium in accordance with another aspect of the present disclosure.

FIG. 14 shows a schematic diagram of a model for sFlt-1 production in accordance with another aspect of the present disclosure.

### DETAILED DESCRIPTION OF THE INVENTION

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a receptor” includes reference to one or more of such receptors, and reference to “the oligomer” includes reference to one or more of such oligomers.

As used herein, “subject” refers to a mammal that may benefit from aspects of the present disclosure. Examples of subjects include humans, and may also include other animals such as horses, pigs, cattle, dogs, cats, rabbits, and aquatic mammals.

As used herein, an “effective amount” or a “therapeutically effective amount” of a substance refers to a non-toxic, but sufficient amount of the substance, to achieve therapeutic, or otherwise desired results in treating a condition for which the substance is thought to be effective. Moreover, an “effective amount” of a non-active agent or drug, such as a carrier, excipients, buffer substance or other component refers to an amount that is suitable to perform a desired role or task, or achieve a desired result. Such amount is generally the minimum amount required, but can be any suitable amount that is considered non-toxic or that would otherwise interfere with the desired function or activity of the formulation or composition in which the ingredient is included. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, the term “treatment” refers to the administration of various dosage forms (for e.g. capsule dosage form) and pharmaceutically acceptable compositions to a subject, or to a change in expression of a molecule in cells or tissue of a subject, who are either asymptomatic or symptomatic. In other words, “treatment” can both be to reduce or eliminate symptoms associated with a condition present in a subject, or it can be prophylactic treatment, i.e. to prevent the occurrence of the symptoms in a subject. Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents (including polynucleotides, polypeptides, etc.) with a carrier or other excipients. Furthermore, the term “dosage form” can include one or more formulation(s) or composition(s) provided in a format for administration to a subject. When any of the above terms is modified by the term “oral” such terms refer to compositions, formulations, or dosage forms formulated and intended for oral administration to subjects. Likewise, when any of the above terms is modified by the term “injectable,” “parenteral,” or “transdermal,” such terms refer to compositions, formulations, or dosage forms intended for such route of administration.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law, “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly, and vice versa. Further, it is to be understood that the listing of components, species, or the like in a group is done for the sake of convenience and that such groups should be interpreted not only in their entirety, but also as though each individual member of the group has been articulated separately and individually without the other members of the group unless the context dictates otherwise. This is true of groups contained both in the specification and claims of this application.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in sonic cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely tack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Invention Embodiments

Soluble vascular endothelial growth factor receptor-1 (sVEGFR-1; also known as sFlt1) is an endogenous regulator of angiogenesis that arises from alternative processing of VEGFR-1 (FLT1). The control of alternative RNA processing is an extremely active area of research, as it underlies proteome diversity and facilitates various post-transcriptional control mechanisms. In the case of FLT1 expression, alternative mRNA processing constitutes a molecular toggle switch—producing functionally distinct molecules, with the soluble receptor acting as a “decoy” that inhibits VEGF angiogenic signaling through membrane-bound receptors. It is shown herein that ribonucleoprotein PTB-binding 2 protein (Raver2; GenBank No. AAH65303.1 UniProt No. Q9HCJ3, incorporated herein by reference) “flips the switch,” shifting FLT1 mRNA processing toward production of the sFlt1 isoform. Raver2 was first described in 2005 (Kleinhenz et al. FEBS 2005. 579: 4254-8) as a likely member of the heterogenous nuclear ribonucleoprotein family, but its biological function to date has been unknown, as has the control mechanisms of FLT1 splicing.

Using murine corneal microarray, the inventors have identified Raver2 as a regulator of sFlt1, Raver2 binds FLT1 mRNA and interacts with polypyrimidine tract-binding protein (PTB) to promote sFlt1 production by inhibiting splicing of at least one key alternatively-processed intron. Raver2-dependent splicing inhibition functionally interacts with telescripting, suggesting that sFlt1-specific processing occurs co-transcriptionally.

Whereas splicing inhibition classically results in exon skipping or redirecting of the splicing machinery to an alternate splice site, these findings support a novel co-transcriptional intron retention mechanism that facilitates cleavage and polyadenylation (C/P) to promote production of sFlt1. It is thus shown that Raver2-dependent regulation of sFlt1 operates in vivo to preserve conical avasularity C57B116 mice. Further, Raver2 suppresses pathologic conical neovascularization in Pax6+/− mice, a welt established model for a blinding human disease, aniridia-related keratopathy (ARK). Additionally, the inventors obtained normal and diseased human corneal specimens and found that Raver2 is highly expressed within normal corneal epithelium but has markedly decreased expression in patients with ARK.

Raver2 loss compromises corneal avascularity whereas overexpression suppresses pathologic conical neovascularization in a model of aniridia-related keratopathy (ARK). Human corneas from patients with aniridia show diminished Raver2 expression, suggesting that Raver2 loss contributes to ARK. The present findings indicate that Raver2 directs RNA processing of FLT1 toward sFlt1, thus preserving conical avascularity.

An elegant regulatory network orchestrates vascular development and angiogenic equilibrium, the disruption of which is pathogenic in many diseases, including cancer, cardiovascular disease, and various blinding disorders. Vascular endothelial growth factor (VEGF) signaling involves a conserved family of angiogenic ligands and receptors, and constitutes a well-studied vascular signaling pathway. sFlt1 has thus emerged as a key endogenous regulator of VEGF signaling that functions in normal vascular development and is dysregulated several diseases of angiogenic imbalance, including preeclampsia, cardiomyopathy, congenital vascular malformation, and cancer. sFlt1 is thus a key preserver of conical avascularity, and loss of sFlt1 is associated with a variety of conditions including, for example and without limitation, pathologic corneal neovascularization (KNV) in aniridia.

As has been described above, production of SFlt1 relies upon alternative RNA processing of FLT1 mRNA wherein C/P take place within an upstream intron, producing a truncated mRNA encoding only the extracellular receptor domain. The truncated receptor functions to quell VEGF signaling by acting as a “ligand sink” and through heterodimerization with membrane-bound VEGFR-1 and VEGFR-2. Expression of the full-length, membrane-bound isoform (mFlt1) requires production of a thirty exon mRNA, wherein sFlt1-specific C/P elements are removed through splicing of intron 13. Thus, intron 13 splicing is a bifurcation point in FLT1 mRNA processing, with retention versus splicing promoting the formation of functionally divergent alternative isoforms sFlt1 or mFlt1, respectively.

Corneal Microarray Identifies Raver2 as Novel Regulator of sFlt1

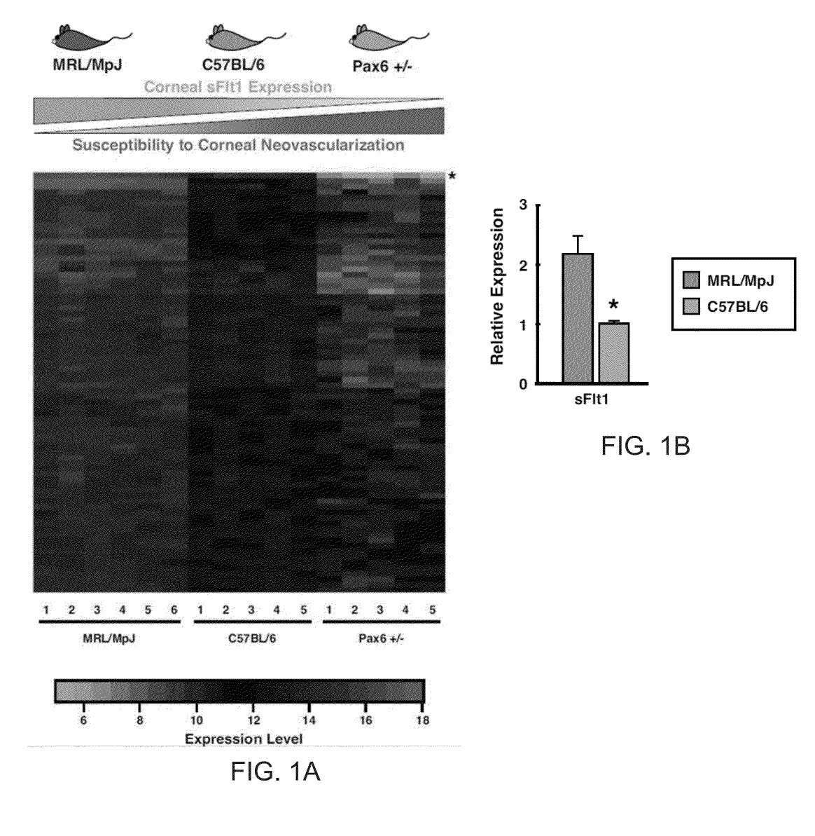

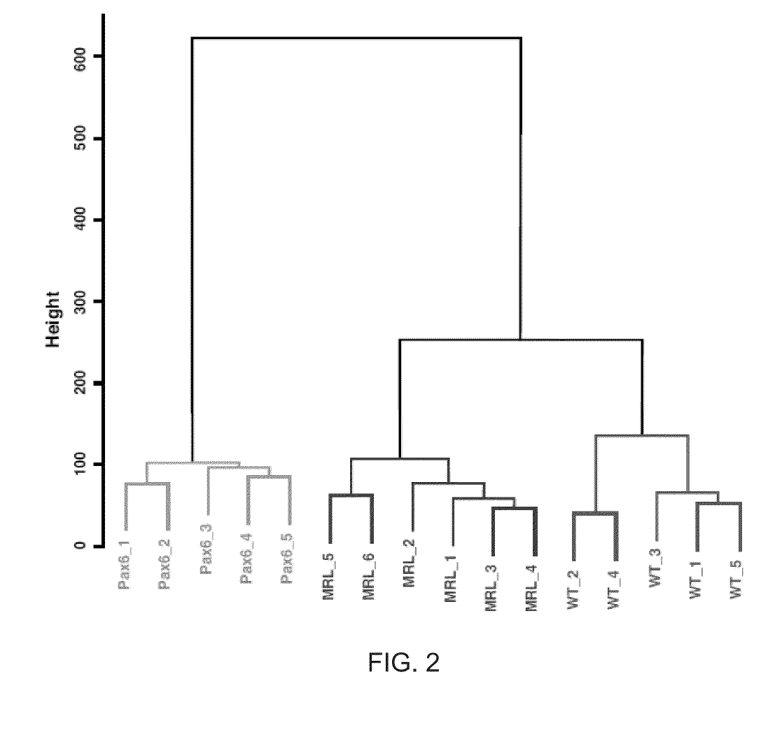

To define novel sFlt1 regulators, the inventors identified three well-characterized mouse strains of common genetic background and with a phenotypic spectrum of susceptibility to KNV: MRL/MpJ mice (known as “healer mice”), which are resistant to KNV; wild-type C57BL/6 mice, which are susceptible to KNV following corneal insult; and Pax6+/− mice, which develop spontaneous KNV. Corneal expression of sFlt1 varies inversely with respect to KNV susceptibility across this spectrum (FIG. 1A,B). It is reasoned that uncharacterized endogenous regulators of sFlt1 may show expression patterns correlated with that of sFlt1 across this model spectrum. Genome-wide mRNA expression microarray analysis was carried out on corneal tissue harvested from MRL/MpJ, C57BL/6, and Pax6+/− mice. Whole genome microarray data clustered tightly among biological replicates of the same strain (FIG. 2). The whole microarray datasets were subjected to unbiased clustering analysis using Ward's method. The data clustered tightly according to mouse strain, demonstrating excellent data quality and reproducibility. MRL/MpJ and C57BL/6 corneal expression patterns were closer to one another than either was to Pax6+/−.

Microarray analysis identified 69 genes with expression highest in MRL/MpJ, intermediate in C57BL/6, and lowest in Pax6+/−, mirroring that of sFlt1. Log (2)-transformed expression data for all biological replicates is shown in FIG. 1A expressed as a heat map for all genes within this group. Each row represents a specific oligonucleotide probe on the array and each column represents an independent biological replicate with strain indicated below the heat map. Genes with expression paralleling sFlt1 were focused on, as these may represent factors that promote sFlt1. The gene with the highest differential expression across the spectrum of corneal models was Raver2, an hnRNP protein with three N-terminal RNA Recognition Motif (RRM) domains (highlighted with an asterisk in FIG. 1A). Quantitative RT-PCR (qRT-PCR) confirmed Raver2 expression was highest in MRL/MpJ, intermediate in C57BL/6, and lowest in Pax6+/− corneas (FIG. 4). Data show corneal expression for n=5 to n=6 biological replicates for each strain. Error bars represent standard deviation, *p<0.05, relative to MRL/MpJ, Student's t test.

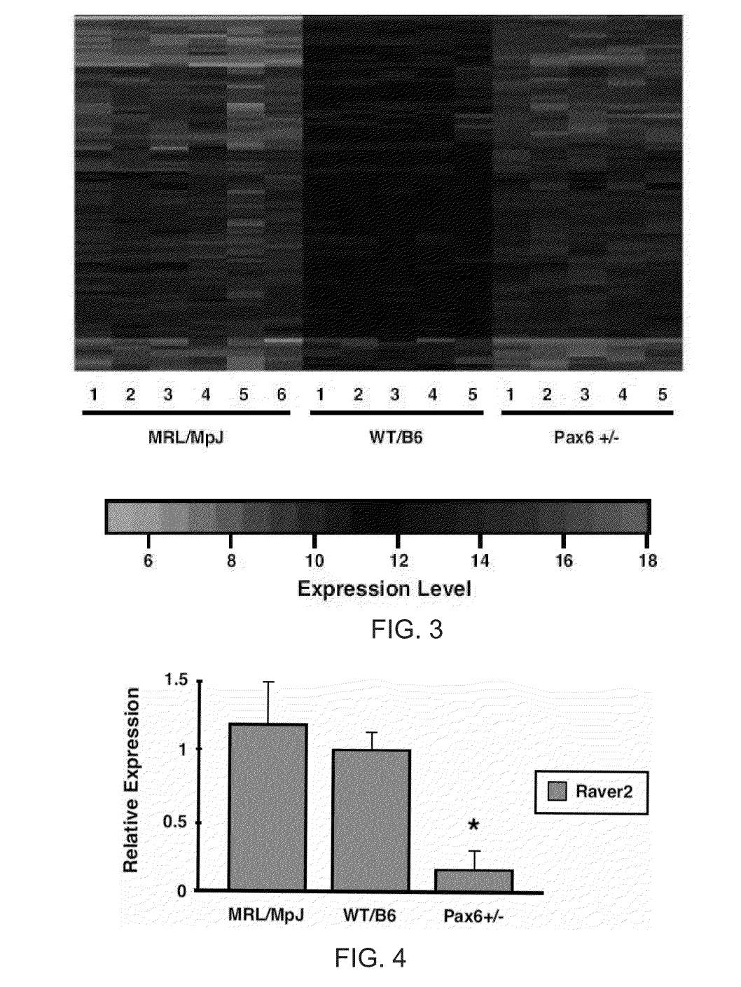

Additionally, 91 genes were identified in the microarray analysis that showed a reciprocal pattern from that described above, as is shown in the heat map of FIG. 3. These genes showed expression lowest in MRL/MpJ, intermediate in C57BL/6, and highest in Pax6+/−. Log (2)-transformed expression data for all biological replicates is expressed as a heat map for all genes within this group. Each row represents a specific oligonucleotide probe on the array and each column represents an independent biological replicate with strain indicated below the heat map.

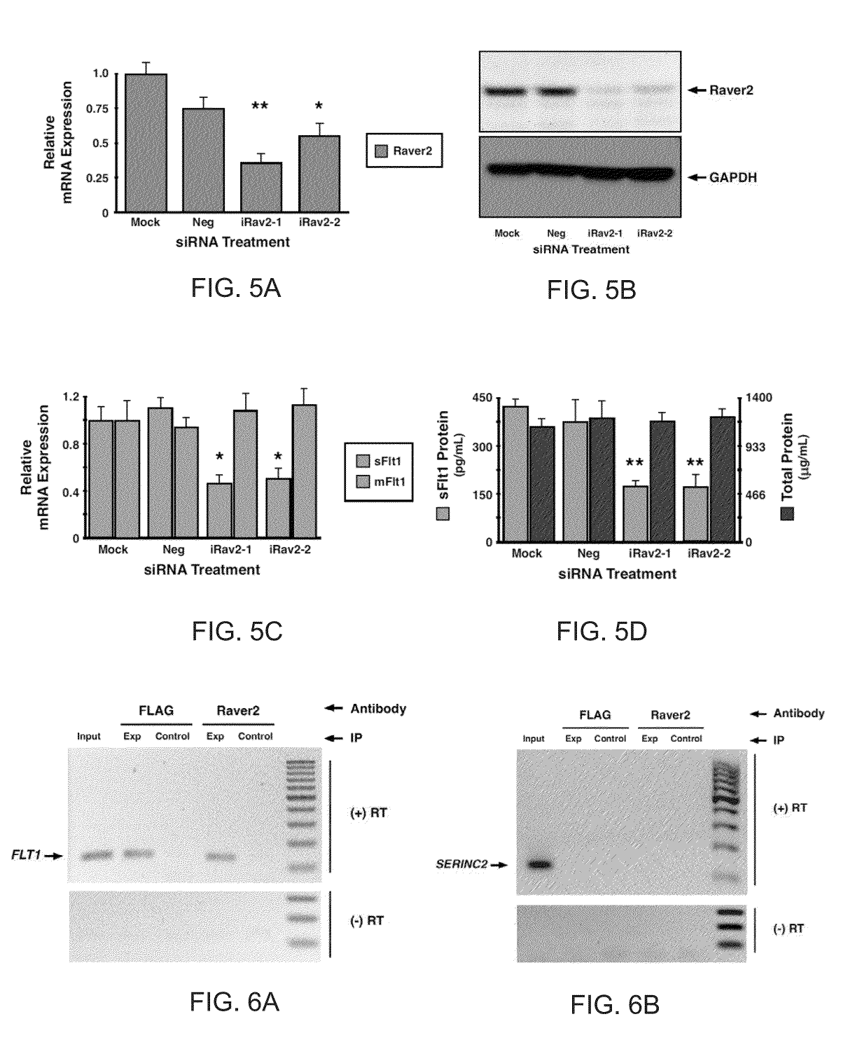

Raver2 previously had no known biological function and was identified as a homologue of another hnRNP, Raver1, based on domain architecture, sequence homology, and similarity within N-terminal RRM domains. As several hnRNPs function mRNA processing and splicing, the combination of a corneal expression profile paralleling sFlt1 and domain structure suggestive of an RNA regulatory actor suggests that Raver2 may promote sFlt1 production. To address this, the inventors first tested whether Raver2 was expressed in human umbilical vein endothelial cells (HUVEC), a relevant vascular line that expresses sFlt1. Western blotting and qRT-PCR demonstrated that Raver2 is expressed in HUVEC. To test if Raver2 influences sFlt1 expression, two independent Raver2-specific small-interfering RNAs (siRNAs) were utilized to knock-down Raver2 HUVEC. FIG. 5A shows quantitative real-time reverse-transcriptase PCR (qRT-PCR) data demonstrating knock-down at the mRNA level, and FIG. 5B shows Western Blotting data demonstrating knock-down at the protein level. Raver2 knock-down in HUVEC resulted in selective decrease of sFlt1 isoform mRNA while mFlt1 isoform mRNA levels trended upward (FIG. 5C). Decreased still expression following Raver2 knock-down was confirmed at the protein level with ELISA analysis of HUVEC culture supernatant (FIG. 5D). Because sFlt1 and mFlt1 share the same upstream regulatory elements and transcriptional start site, it is unlikely that the difference in isoform expression is due to transcription-level changes. These findings demonstrate that Raver2 promotes expression of sFlt1 HUVEC, and strongly suggest that Raver2 acts at a post-transcriptional level to regulate sFlt1 production.

The Observation that Raver2 selectively promotes sFlt1 production raised the possibility that it modulates mRNA processing, the bifurcation point in FLT1 expression. If so, it was predicted that Raver2 would interact with FLT1 mRNA. Several lines of evidence indicate Raver proteins can bind RNA. Isothermal titration calorimetry demonstrated that the Raver1 RRM1 domain binds RNA with micromolar affinity and fluorescence resonance energy transfer (FRET) studies demonstrated binding of a full-length YFP-Raver1 fusion protein to nuclear RNAs in situ. Furthermore, ribohomopolymer binding assays demonstrate that an N-terminal Raver2 fragment containing all three RRM domains is capable of binding to G-rich RNA polymers. To determine if Raver2 could associate with endogenous FLT1 mRNA, the inventors performed RNA immunoprecipitation (RIP) in HUVEC expressing FLAG-tagged Raver2. Immunoprecipitation was performed using both FLAG monoclonal and Raver2 polyclonal antibodies, providing two independent mechanisms for enriching Raver2-associated RNA. RIP was performed in HUVEC expressing FLAG-tagged Raver2 with both anti-FLAG and anti-Raver2 antibodies. Following IP with experimental or control antibodies, associated RNA was analyzed by reverse-transcriptase PCR (RT-PCR), demonstrating localization of Raver2 to FLT1 mRNA (FIG. 6A), but not to a control mRNA, SEM (FIG. 6A, B). (Data report mean of n=4 independent replicates with error bars representing standard deviation. *p<0.05, **p<0.01 relative to control, Student's t test.) As such, gene-specific RT-PCR showed that FLT1 mRNA is enriched following RIP with Raver2 or FLAG antibody, but not with control antibody. Primers targeting a control mRNA failed to show any enrichment following Raver2 IP. These results show that Raver2 binds endogenous FLT1 mRNA, localizing it to the critical substrate for FLT1 isoform processing.

PTB Interacts with Raver2 and Binds FLT1 mRNA in a Raver2-Dependent Fashion

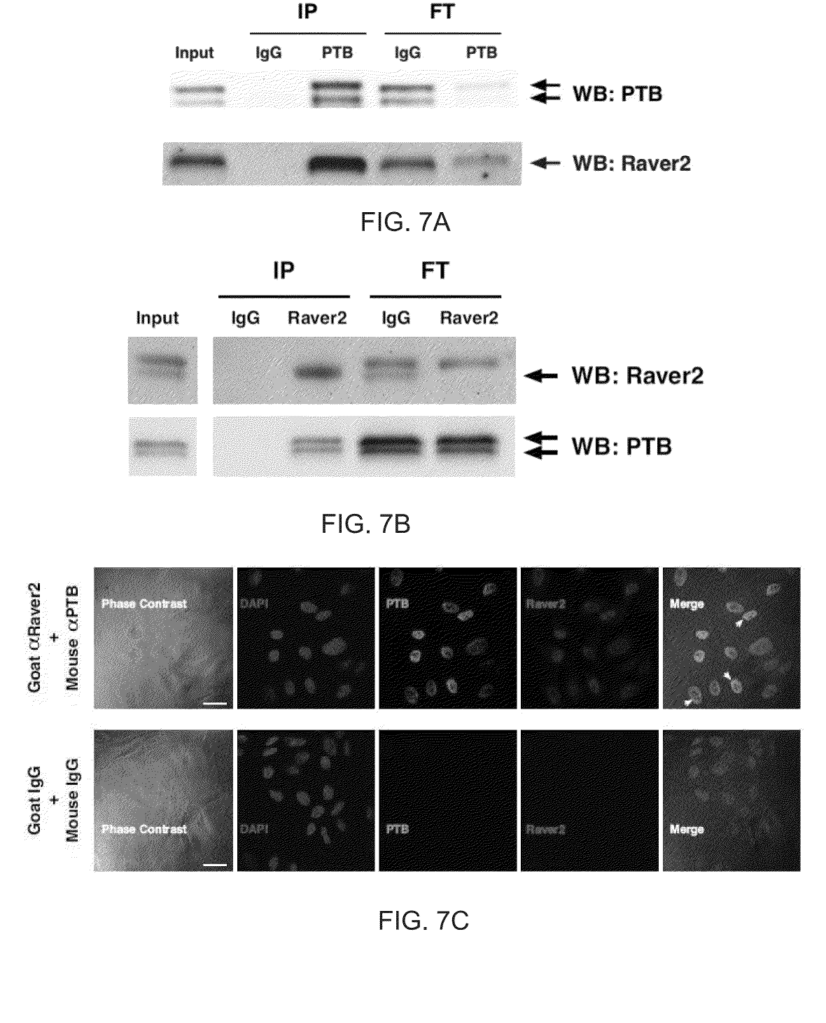

It was next explored how Raver2 might regulate Furl mRNA processing. The homologue Raver1 is a binding partner fir polypyrimidine tract binding protein (PTB). Crystallographic and targeted mutational studies have mapped the Raver1-binding domain of PTB and the PTB-binding segments of Raver1. Similar to Raver1, it is possible that Raver2 can bind PTB via conserved Raver peptide motifs. As PTB is a well characterized RNA-binding factor modulating post-transcriptional mRNA processing, the inventors examined whether a Raver2-PTB interaction can regulate FLT1 mRNA processing. First, it was tested if Raver2 interacts with PTB in HUVEC cells. Immunoprecipitation (IP) experiments demonstrate co-IP of Raver2 following PTB IP (FIG. 7A; Western Blot, HUVEC cell lysate) and co-IP of PTB following Raver2 IP (FIG. 7B). Furthermore, immunofluorescence studies in HUVEC cells demonstrate nuclear colocalization of Raver2 and PTB (FIG. 7C). FIG. 7C shows immunofluorescence using antibodies specific for PTB and Raver2 with 4′,6-diamidino-2-phenylindole (DAPI) nuclear staining. Staining with isotype antibody controls is shown in lower panel. Both PTB and Raver2 demonstrate strong nuclear staining, but Raver2 also has some signal within the cytoplasm. Merge (far right) demonstrates PTB and Raver2 colocalization within HUVEC nuclei.

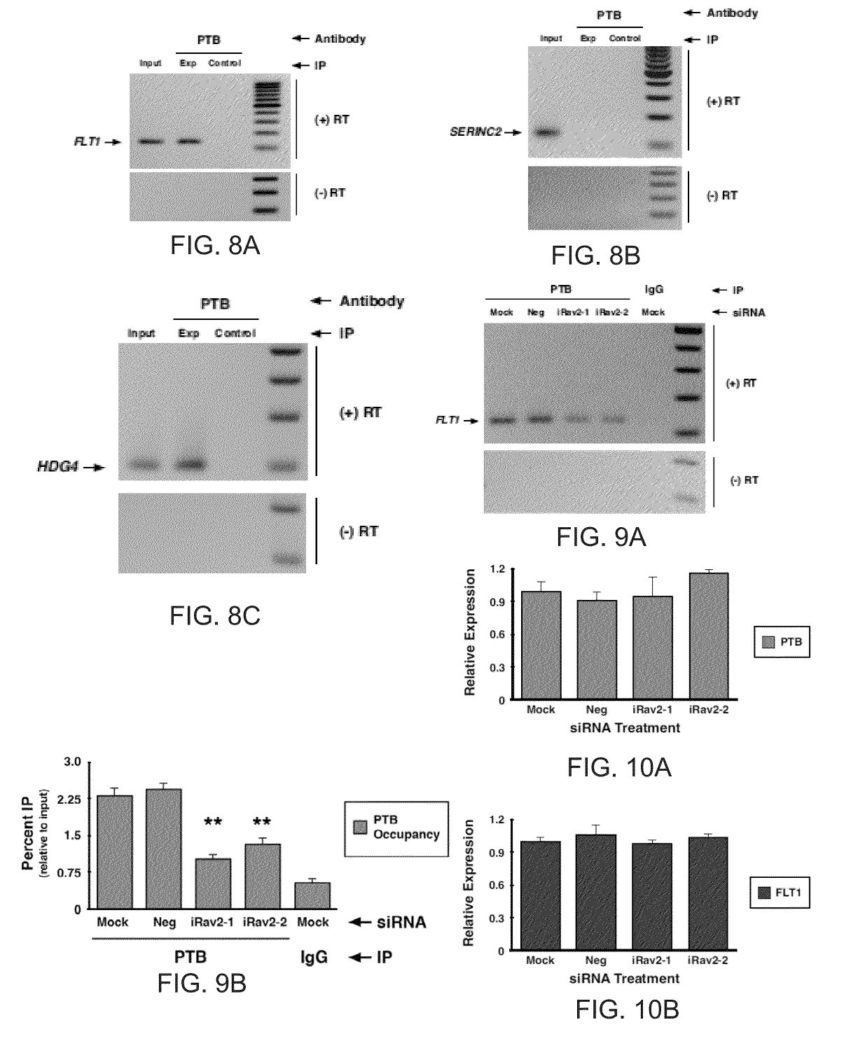

If Raver2 and PTB cooperatively regulate FLT1. mRNA processing, it is expected that PTB localizes to FLT1 mRNA, similar to Raver2. To test this, the inventors performed PTB RIP from HUVEC cells followed by reverse transcriptase PCR (RT-PCR) and found that FLT1 mRNA is enriched following IP with PTB monoclonal antibody, but not with control antibody (FIG. 8A). Primers targeting a negative control mRNA (SEA 1) failed to show any enrichment (FIG. 8B). Positive control loci (HDG4) verified the quality of the PTB RIP (FIG. 8C), with control PTB target loci based on recent genome-scale CLIP-seq mapping in HeLa cells. Together, these results demonstrate that Raver2 interacts with PTB and that both factors bind endogenous FLT1 mRNA.

The interaction between Raver proteins and PTB raises the possibility that Raver1 and/or Raver2 may stabilize PTB assembly on endogenous RNAs. To test if Raver2 is necessary for PTB localization to FLT1 mRNA, the inventors performed PTB RIP following Raver2 knock-down. Gene-specific RT-PCR showed that PTB occupancy at FLT1 nRNA decreases following Raver2 knock-down (FIG. 9A). Loss of PTB localization was confirmed by qRT-PCR analysis of RIP eluates (FIG. 9B). (Data report mean of n=4 independent replicates with error bars representing standard deviation. **p<0.01 relative to control, Student's t test.) Interestingly, decreased PTB occupancy at FLT1 mRNA following Raver2 knock-down was not due to changes in PTB levels or loss of FLT1 mRNA available for binding (FIG. 10A,B). FIG. 10A shows qRT-PCR in HUVEC following Raver2 knockdown demonstrating no significant change in PTB expression. Regarding FIG. 10B, primers utilized in PTB RIP were used for qRT-PCR using random-hexamer primed cDNA following Raver2 knock-down in HUVEC, showing no significant change in FLT1 pre-mRNA levels. Data report mean of n=4 independent replicates, error bars represent standard deviation. It is noted that control experiments and RIP assays utilized random hexamer-primed cDNA (allowing for amplification of nascent mRNA that has not yet been polyadenylated), whereas experiments addressing production of mature, polyadenylated FLT1 mRNA isoforms utilized oligo(dT) primed cDNA. These results demonstrate Raver2 is required for PTB localization to FLT1 mRNA in HUVEC cells, and suggest Raver2 and PTB cooperatively regulate FLT1 mRNA processing.

Raver2 Inhibits Splicing of Alternatively Processed Intron 13

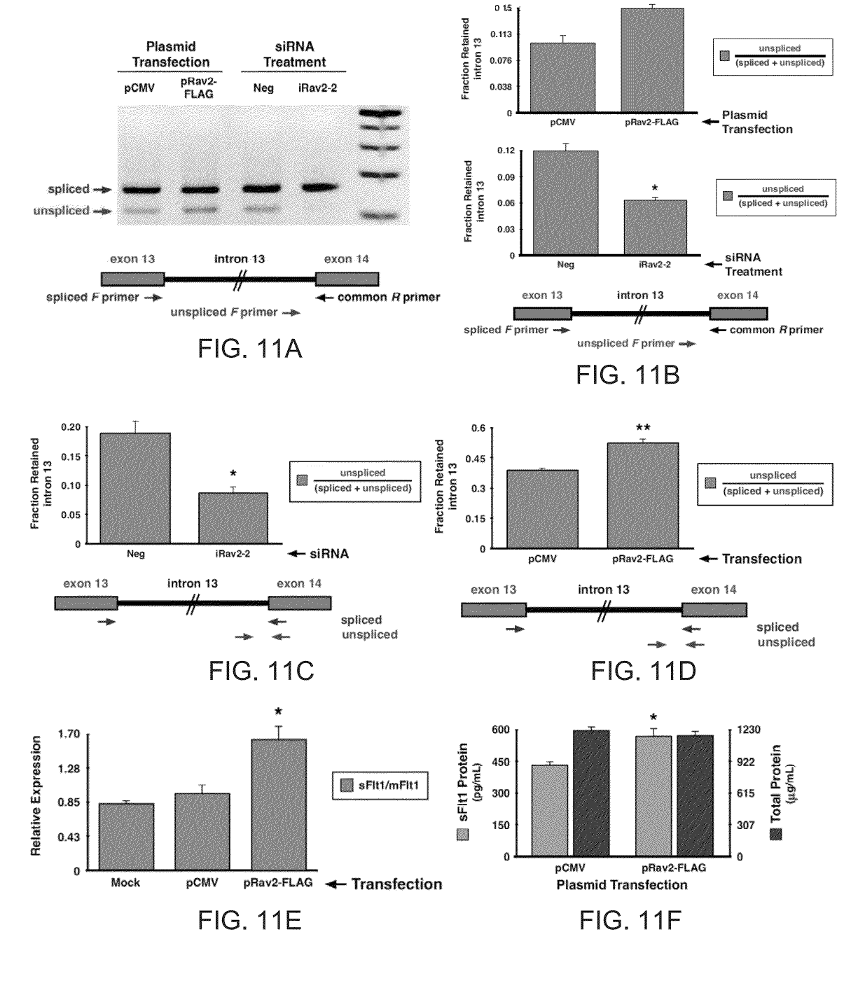

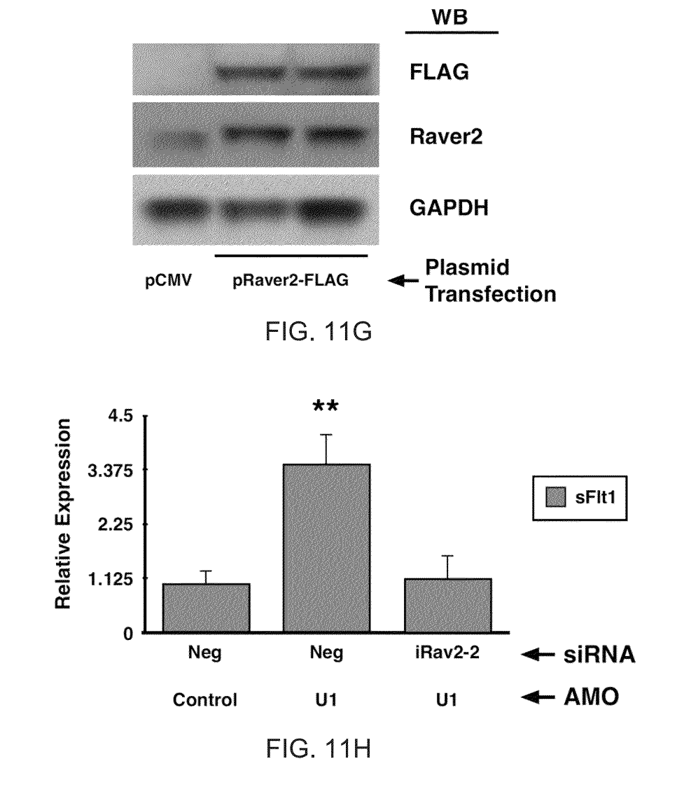

White PTB regulates diverse steps in post-transcriptional RNA processing, one principal function is to regulate repression of alternative splicing. Minigene assays in cell culture systems have shown that Raver1 can act as a PTB-associated co-repressor to inhibit splicing of alternative cassette exons. The inventors hypothesized that Raver2/PTB may repress splicing of FLT1 intron 13, which would facilitate retention of sFlt1-specific sequence elements and promote production of the truncated isoform. To test whether Raver2/PTB regulates intron 13 splicing, the inventors designed three-primer PCR reactions containing two forward primers (one upstream exonic and one intronic) and a common reverse primer (downs(ream exonic), to simultaneously amplify unspliced and spliced templates. The relative amount of each product reflects the degree of intron 13 retention versus splicing for endogenous FLT1 mRNA. Random-hexamer primed cDNA was used as template, allowing for analysis of precursor in RNAs that have not yet undergone polyadenylation. Following knock-down of Raver2 in HUVEC cells (which results in loss of PTB occupancy at FLT1 mRNA), the level of intron 13-containing mRNAs decreased, indicating activation of exon 13 to exon 14 splicing (FIG. 11A, compare lanes 3 and 4). Conversely, overexpression of Raver2 increased intron 13-containing mRNAs, indicating inhibition of exon 13 to exon 14 splicing (FIG. 11A, compare lanes 1 and 2). Primer positions are shown in schematic diagram below gel. Quantitative densitometry of three primer PCRs for multiple independent biological replicates showed similar results (FIG. 11B). Densitometric analysis of spliced and unspliced PCR products shows that Raver2 overexpression increased intron 13 retention, whereas Raver2 knock-down had the reciprocal effect. Primer positions are shown in schematic diagram at the bottom of FIG. 11B. Data report mean of n=3 to n=4 independent replicates for each group, error bars represent standard error of the mean. *p<0.05 relative to control, Student's t test.

To confirm that alteration of Raver2 expression affected intron 13 splicing, the inventors performed qRT-PCR with separate primer sets specific for unspliced versus spliced template, again using random-hexamer primed cDNA. Raver2 knock-down or overexpression enhanced or inhibited exon 13 to exon 14 splicing, respectively (See FIGS. 11C,D). Thus, Raver2 knock-down decreased intron 13 retention, consistent with enhanced splicing (FIG. 11C) and Raver2 overexpression increased intron 13 retention, consistent with splicing inhibition (FIG. 11D). Taken together, this data is consistent with a model wherein Raver2 and PTB cooperatively inhibit splicing of the key alternatively processed intron 13. Importantly, inhibition of intron 13 splicing following Raver2 overexpression increases sFlt1 relative to mFlt1 (FIG. 11E; qRT-PCR), and enhances sFlt1 production (FIG. 11F; ELISA). These results link Raver2-mediated inhibition of intron 13 splicing to isoform-specific upregulation of sFlt1. FIG. 11G shows a Western blot for Raver2 and FLAG demonstrating overexpression of Raver2-FLAG HUVEC cells transfected with pRaver2-FLAG relative to vector control.

Gene expression requires coordination among processes that are spatially and temporally linked, including transcription, splicing, and C/P. Interactions between splicing and transcription are well established, and recent studies have revealed that premature C/P is broadly suppressed by a conserved U1 snRNP-dependent co-transcriptional mechanism termed telescripting. Because of its snRNP-dependence, telescripting can be blocked and C/P de-repressed using U1-specific antisense morpholino oligonucleotides (AMO). The inventors have utilized this system to investigate C/P activity at intron 13, by targeting U1-AMO to the FLIT exon 13/intron 13 junction, which enhances sFlt1 production through intronic C/P de-repression. It was then tested if Raver2 affects the availability of intronic sFlt1-specific elements by pre-treating with Raver2-specific siRNAs. HUVEC cells pre-treated with control siRNA followed by U1-specific AMO showed enhanced sFlt1 production (FIG. 11H, compare first and second bars), whereas pre-treatment with Raver2-specific siRNA blocked this response (FIG. 11H, compare second and third bars). Regarding FIG. 11H, sequential siRNA/AMO treatment in HUVEC was performed. Cells were first transfected with the indicated siRNA, incubated for 48 hours, then transfected with AMO and incubated for an additional 24 hours. U1 AMO treatment increased sFlt1 production following control siRNA treatment, but this effect was blocked by pre-treatment with Raver2-specific siRNA. Data report mean of n=3 to n=6 independent replicates with error bars representing standard deviation. *p<0.05, **p<0.01 relative to control, Student's t test. These data demonstrate a functional interaction between Raver2-dependent splicing inhibition and U1-mediated telescripting activity at FLT1 intron 13, and suggest that following Raver2 knock-down, enhanced exon to exon 14 splicing leaves intronic elements less available for processing by the C/P machinery. Moreover, the dominant effect of Raver2 knock-down over U1 AMO treatment suggests that Raver2 acts co-transcriptionally, as U1 AMO treatment would be expected to supersede the effects of Raver2 knock-down if Raver2 acted later in RNA processing.

Raver2 Regulates Corneal Avascularity

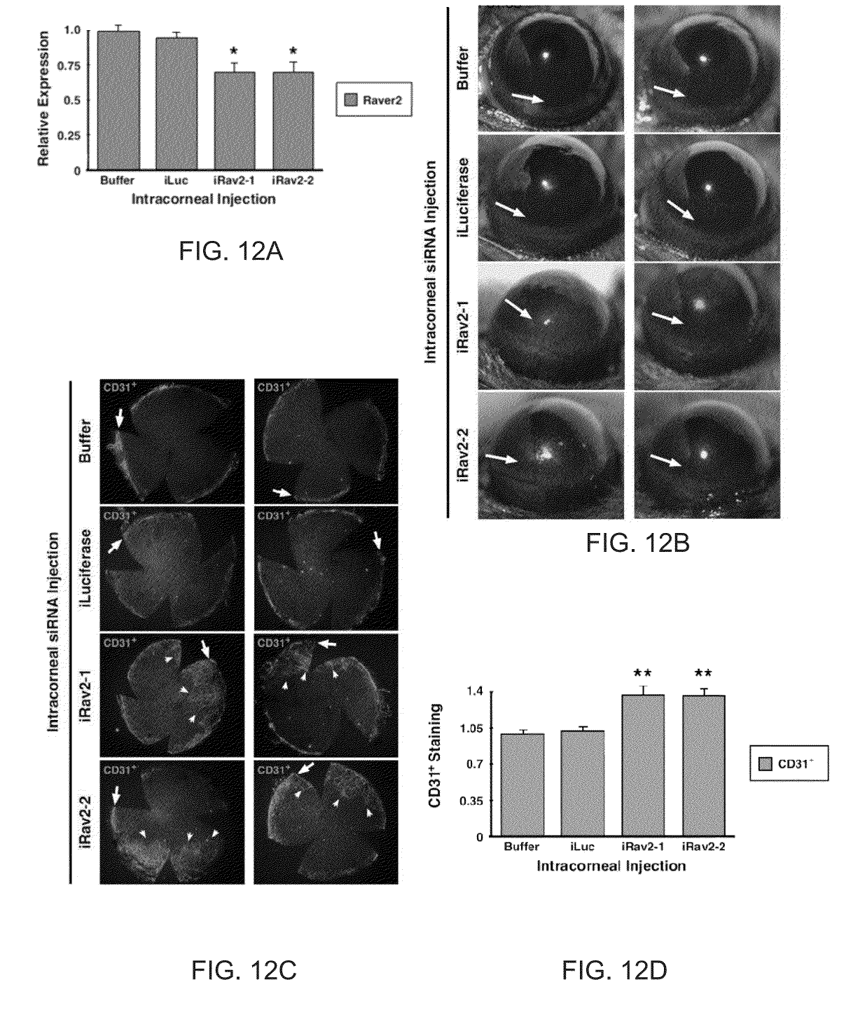

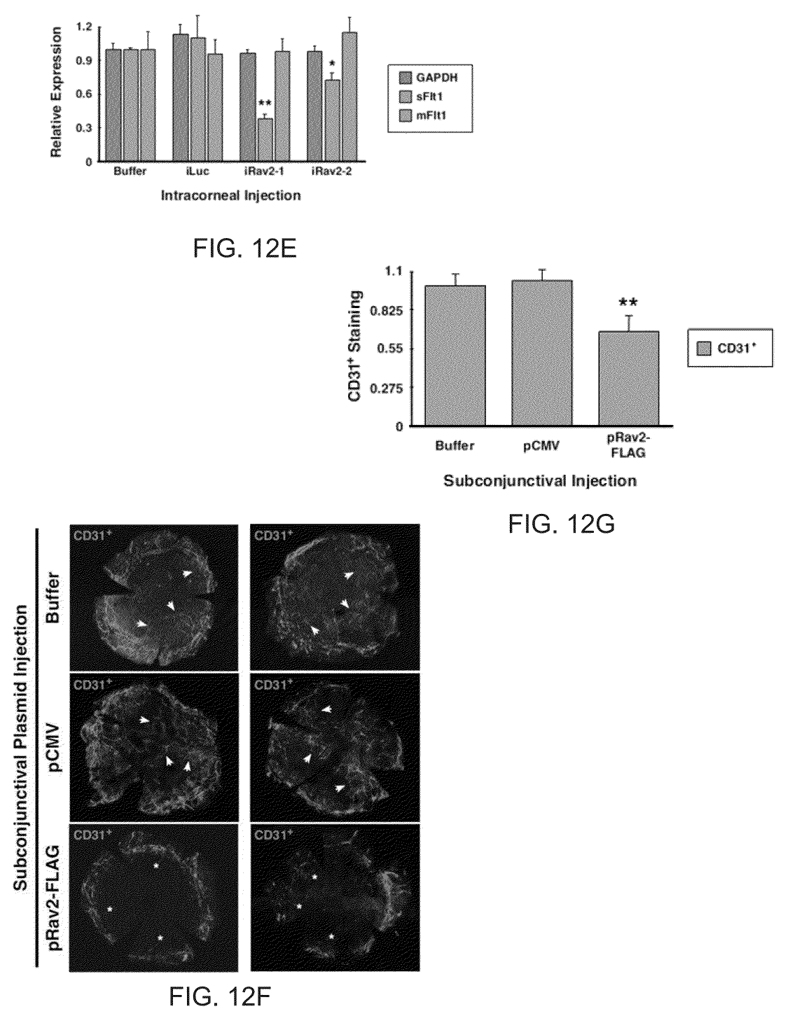

As the inventors initially identified Raver2 within a spectrum of clinically relevant models of corneal neovascularization, it was explored if the mechanistic insights obtained in vitro are operative in animal models. To test for cornea-specific functions of Raver2, the inventors injected plasmid bearing Raver2-specific siRNAs into the corneal stroma of wild-type C57BL/6 mice. Raver2 knock-down within the cornea (FIG. 12A) was accompanied by marked KNV (FIG. 12B bottom two rows; FIG. 12C), whereas corneas injected with control plasmid or buffer remained avascular (FIG. 12B, top two rows; FIG. 12C). FIG. 12B shows representative photographs of C57BL/6 mouse corneas 14 days after intracorneal injection. Arrows indicate the normally avascular area of the cornea immediately central to the timbal vascular arcade. This area remains avascular following injection of buffer or negative control siRNA (upper two panels), but undergoes marked. KNV following injection of Raver2-specific siRNAs (lower two panels). FIG. 12C shows representative flat-mounts of C57BL/6 corneas fourteen days following intracorneal injection of buffer or iLuciferase siRNA control (upper panels) compare(to injection of two distinct Raver2-specific siRNAs (lower panels) as in FIG. 12B. Intracorneal Raver2 knock-down induced marked KNV, evidence by prominent CD31+ blood vessels (marked by white arrowheads, lower panels) located well beyond the normal limbal arcade (white arrow). Quantification of corneal CD31+ immunofluorescence (central to the limbal arcade) demonstrates significant corneal neovascularization C57BL/6 eyes following intracorneal Raver2 knock-down, as shown in FIG. 12D. Data report mean of n=3 to n=6 independent replicates for each treatment group with error bars representing standard deviation. **p<0.01 compared to control, Student's t test. Furthermore, Raver2 knock-down in vivo correlates with specific down regulation of sFlt1, with mFlt1 showing no significant expression change (FIG. 12E). Taken together, these results demonstrate that Raver2 is required for corneal avascularity through isoform-specific regulation of FLT1 expression. Regarding FIG. 12E, qRT-PCR shows that KNV following Raver2 knock-down in C57BL/6 corneas is linked to decreased expression of sFlt1, while the mFlt1 isoform trends toward increased expression (not statistically significant) and a control gene, GAPDH, remains unchanged.

The inventors hypothesized that if Raver2 knock-down compromises corneal avascularity wild-type mice, overexpression of Raver2 may prevent pathologic spontaneous Pax6+/− mice, a murine correlate of human aniridia-related keratopathy (ARK), in which Pax6 gene defects are associated with vision threatening spontaneous KNV. To determine if Raver2 overexpression can suppress KNV Pax6+/− mice, juxtacorneal subconjunctival microinjection of plasmids encoding Raver2 were performed, which suppressed KNV Pax6+/− eyes; whereas control injections of buffer or empty plasmid showed no effect (FIGS. 12F,G). Hence, overexpression of Raver2 prevents spontaneous KNV in a well characterized model of a blinding human disease, ARK. Regarding FIG. 12F, representative flat-mounts are shown of Pax6+/− corneas, a well-established model of aniridia-related keratopathy. Seven days following control juxtacorneal subconjunctival injection of either buffer or empty vector, Pax6+/− eyes acquire KNV, evidenced by prominent CD31+ blood vessels located well beyond the timbal arcade (arrowheads, upper two panels). KNV is markedly attenuated in Pax6−/+ eyes receiving similar injection of a plasmid bearing Raver2, with blunted vessels seen near the timbal arcade (asterisks, lower panel). Regarding FIG. 12G, quantification of corneal CD31+ immunofluorescence (central to the limbal arcade) demonstrates significant reduction of abnormal conical neovascularization Pax6+/− eyes following subconjunctival injection with pRaver2-FLAG compared to treatment with buffer or empty vector. Data report mean of n=3 to n=6 independent replicates for each treatment group with error bars representing standard deviation. *p<0.05, **p<0.01 compared to control, Student's t test.

Pax6+/− murine corneas, low levels of Raver2 likely underlie the previously described tow levels of sFlt1. Given that Pax6+/− corneas recapitulate many features of aniridia, and corneas from human patients with ARK show decreased expression of sFlt1, the inventors tested if corneal expression of Raver2 was altered in ARK. Human corneal specimens from normal donors and patients with aniridia were analyzed using immunohistochemistry to determine Raver2 expression levels. Normal corneas showed strong Raver2 expression localized primarily within corneal epithelium (FIGS. 5,A and B), whereas aniridia specimens showed diminutive Raver2 expression (FIGS. 5,C and D). These results demonstrate that Raver2 is expressed in normal human corneal tissue, and suggest that diminished Raver2 expression may contribute to the pathogenesis of ARK.

Raver2 is expressed at high levels in normal human corneal epithelium and diminished in patients with aniridia-related keratopathy (ARK).

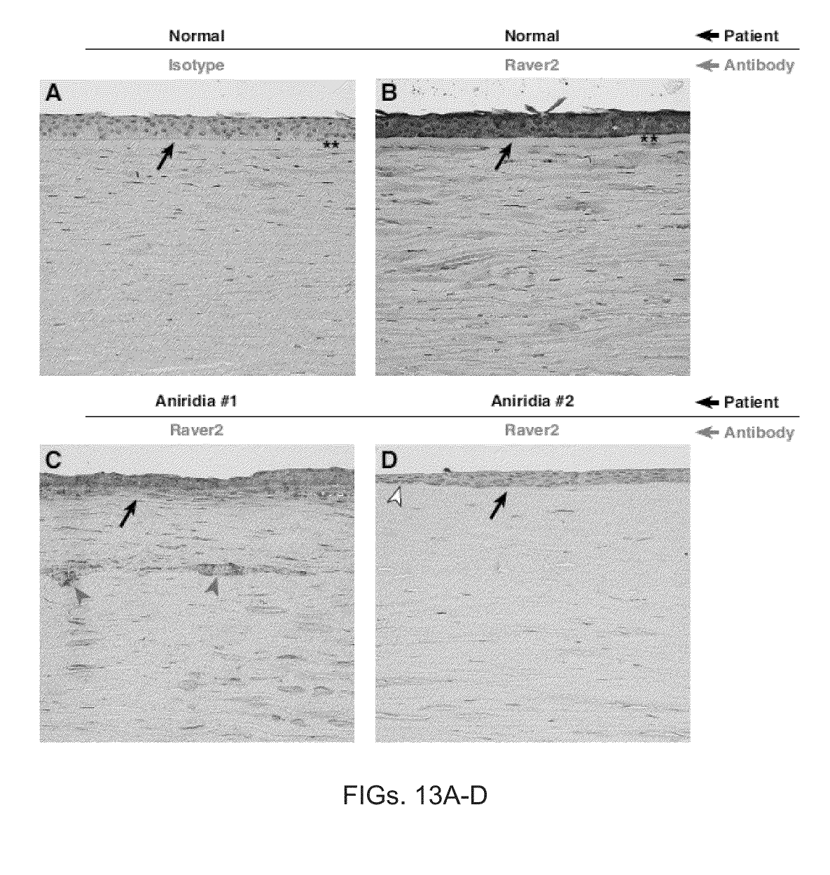

FIGS. 13A-D show images of inummohistochemical staining of normal human cornea. Raver2-specific (FIG. 13B) antibody demonstrates strong staining within the corneal epithelium (arrow), whereas no signal is seen using isotype control antibody (FIG. 13A). Normal Bowman's membrane is clearly visible (at **) as an acellular band located between the corneal epithelium and corneal stroma. FIGS. 13C, D show immunohistochemical staining of human aniridia cornea specimens removed at the time of corneal transplantation. Raver2-specific staining is significantly reduced within corneal epithelium (arrows). Specimens show hallmarks of ARK including vascularization (red arrowheads), epithelial thinning (white arrowhead), and lack of regular Bowman's membrane (compare to FIGS. 13A and B). All slides used hematoxylin counterstain, magnification is 1.0× for all photomicrographs.

One Possible Model for Raver2/PTB-mediated sFlt1 Production.

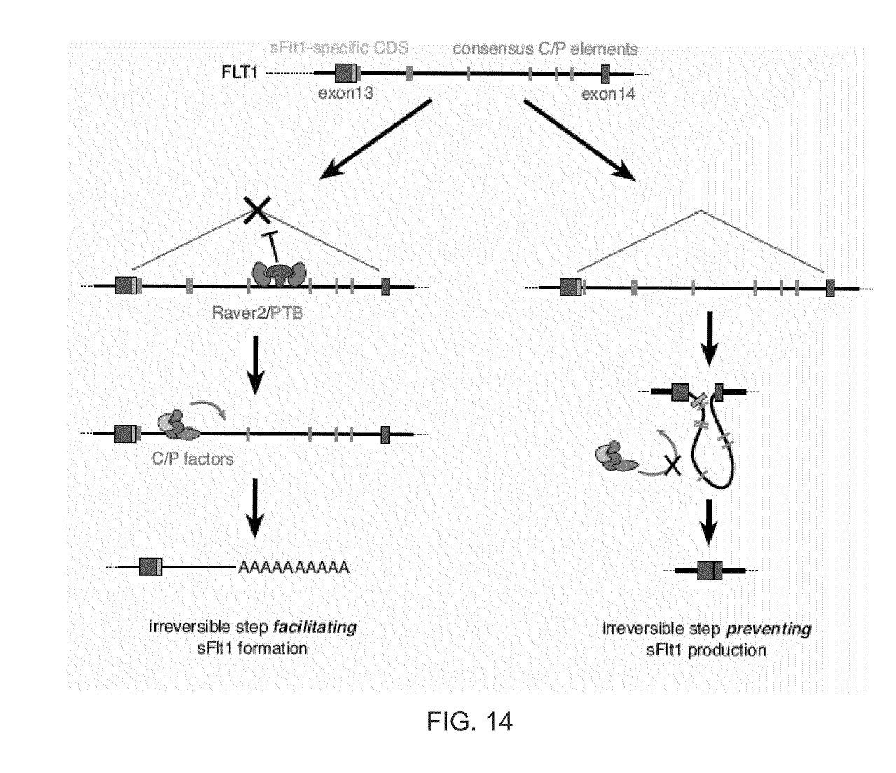

FIG. 14 shows a schematic diagram of FLT1 showing intron 13 (black line) and flanking exons (boxes), sFlt1-specific coding sequence is shown in orange and consensus cleavage and polyadenylation (C/P) sequence elements are labeled as vertical lines. (Left) In HUVEC cells and wild-type corneal tissue. Raver2/PTB binding to FLT1 mRNA inhibits exon 13 to exon 14 splicing, resulting in retention of intron 13. This leaves C/P elements available for processing by the C/P machinery, resulting in an irreversible step toward sFlt1 production. (Right) When Raver2 is limiting following knock-down or in Pax6+/− corneal tissue, Raver2/PTB occupancy decreases and exon 13 to exon 14 splicing is enhanced, resulting in irreversible removal of sequence elements required for sFlt1 production.

### Discussion

The inventors have thus utilized a system of KNV models to identify Raver2 as a novel promoter of sFlt1, a clinically important endogenous regulator of VEGF signaling. While previous studies have identified upstream signal transduction factors that modulate FLT1 expression, the present data reveals that Raver2 is a direct and specific regulator of sFlt1. Raver2 likely aids in recruiting and/or stabilizing PTB assembly on FLT1 mRNA, where the two factors act in concert to repress splicing of the key alternatively processed intron 13 to enable intron retention and early polyadenylation. Minigene assays have identified multiple sequence elements within intron 13 that promote intronic C/P. It is thus proposed that Raver2/PTB-mediated splicing inhibition leads to co-transcriptional intron retention, leaving these elements available for processing by the C/P machinery, tipping the balance of FLT1 expression toward the sFlt1 isoform (FIG. 14). Intron retention is an uncommon and unique mode of alternative splicing distinguished by a lack of exon-skipping or competing splice sites, and best characterized in retroviral genome expression. However, intron retention can regulate the expression of cellular genes through modulation of subcellular localization and/or translation. While these examples of “permanent” intron retention involve production of intron-containing mature mRNAs, the present data suggests that in FLT1 processing, a distinct co-transcriptional intron retention mechanism functionally interacts with another RNA processing pathway, U1-mediated telescripting, to influence mRNA isoform choice. Recent evidence that PTB can bind directly to U1 snRNA corroborates the potential for direct interaction between these pathways.

While intronic C/P is an uncommon mechanism for generating alternative mRNA isoforms, several genes have an architecture resembling FLT1 and can produce stable truncated isoforms through intronic C/P. These include other receptor tyrosine kinases, immunoglobulin genes, and certain neuronal genes. While telescripting likely plays an important repressive role at these loci, no endogenous factor(s) have been identified that promote RNA processing toward the truncated isoform. It is possible that co-transcriptional intron retention may be a common regulatory mechanism promoting intronic C/P, and factors such as Raver2/PTB may inhibit splicing at key introns to promote production of stable truncated isoforms at other loci.

The Observation that Raver2 is both required for avascularity in C57BL/6 corneas and expressed at high levels in normal human corneal epithelium suggests that Raver2 has an evolutionarily conserved role in preserving corneal avascularity. In Pax6+/− murine corneas and human patients with aniridia, tow levels of Raver2 likely underlie the previously described tow levels of sFlt1 and thus contribute to the pathogenesis of vision-threatening ARK. The observation that Raver2 overexpression suppresses KNV in Pax6+/− mice identifies Raver2 as a therapeutic target for ARK, as well as other corneal neovascular disorders. Furthermore, the growing number of human diseases deriving from sFlt1 dysregulation suggests that insights into FLT1 processing may be broadly applicable.

Accordingly, a method of treating a condition in a subject resulting from abnormally high VEGF signaling through membrane-bound receptors is provided. In one aspect such a method can include controlling expression of Raver 2 in a subject, including either systemically or in selected anatomical locale, region, or location of a subject. In one example, such a method may be implemented by administering or otherwise increasing or decreasing expression of Raver2 in affected cells of the subject to increase production of soluble VEGF receptors. In another aspect, administering or otherwise increasing expression of Raver2 decreases production of membrane-bound VEGF receptors one specific example, the soluble VEGF receptor is sFlt-1 and the membrane-bound VEGF receptor is mFlt-1.

More specifically, a method of treating a condition resulting from abnormally high VEGF signaling through membrane-bound VEGF receptors can include administering to a subject in need of such treatment an effective amount of a polypeptide having a sequence region with at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or an appropriate combination thereof. It is noted that “administering” can include a variety of actions that result in the increase of the polypeptide in the subject, including polypeptide delivery, stimulating polypeptide production and/or expression, and the like. The polypeptide can thus upregulate soluble VEGF receptor production in affected cells to decrease the abnormally high VEGF signaling through the membrane-bound VEGF receptors. In some aspects the sequence region includes the all or substantially all of the polypeptide sequence. In other aspects, the sequence region include only a portion of the complete polypeptide sequence. In yet another aspect, the sequence region can have 100% sequence identity to at least one of SEQ ID 001, SEQ ID 002, SEQ ID 003, SEQ ID 004, SEQ ID 005, SEQ ID 006, SEQ ID 007, SEQ 008, SEQ ID 009, SEQ ID 010, SEQ ID 011, or an appropriate combination thereof. In yet another aspect, the polypeptide is Raver2. In yet a further aspect, the polypeptide has at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, or SEQ ID 011. In another aspect, the polypeptide has 100% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011.

It is additionally contemplated that, in some aspects, a polynucleotide can be utilized to administer the effective amount of the polypeptide to the subject. A polynucleotide can include type of polynucleotide or biomolecule comprising nucleotide monomers, such as, for example, DNA, RNA, mRNA, cDNA, and the like. The polynucleotide can thus be used to generate the poly peptide once inside cells of the subject. In one aspect, for example, administering the effective amount of the polypeptide further includes administering an effective amount of a poly nucleotide encoding the polypeptide or polypeptide region, wherein the polynucleotide has at least 85% sequence identity to SEQ ID 012. In another aspect, the polynucleotide has at least 90% sequence identity to SEQ ID 012. In yet another aspect, the polynucleotide has at least 95% sequence identity to SEQ ID 012. In a further aspect, the polynucleotide has 100% sequence identity to SEQ ID 012. It is noted that, in cases where only a portion of the polypeptide is to be encoded, a portion of the polynucleotide encoding the portion of the polypeptide can be utilized.

A variety of conditions are contemplated to be treated, and any such condition that can be effectively treated via Raver2 is included in the present scope. General non-limiting examples can include cancer, macular degeneration, diabetic retinopathy, rheumatoid arthritis, corneal injury, conical transplant rejection, or combinations thereof. In another aspect, the condition can include an ocular condition. Non-limiting examples can include macular degeneration, diabetic retinopathy, corneal injury, corneal transplant rejection, and the like, including appropriate combinations thereof is noted that the present scope additionally includes the prevention of any condition for which Raver2 can be used as a treatment. For example, it is contemplated that Raver2 can be administered or its expression can be increased or decreased in an individual at risk for a condition, whether imminent or not. In some cases, such an individual can be undergoing a procedure such as an intrusive ocular surgery where the increase in Raver2 administration can function to prevent or minimize corneal corneal transplant rejection, or the like.

It is noted that any technique for administering and/or increasing or decreasing expression of Raver2 in an individual is included within the present scope. Non-limiting examples can include various administered foundations, expression vectors such as plasmids, adeno-associated virus (AAV), and the like, small molecule agonists, proteins, biologically active protein fragments, and the like, including appropriate combinations thereof.

In one aspect, for example, a polynucleotide that encodes Raver2 or a fragment of Raver2, such as a sequence region, can be utilized in the administration or modification of expression. Any technique or construct useful for delivering or expressing such a polynucleotide is considered to be within the present scope. In one aspect, for example, an expression vector containing the polynucleotide can be introduced or otherwise administered to the subject, either systemically or to a localized region of cells or tissue.

The term “expression vector” is well known in the art, and can refer to anon-viral or a viral vector that includes the polynucleotide encoding the polypeptide (e.g., Raver2) in a form suitable for expression of the polynucleotide in a host cell of the subject. A plasmid is a common type of non-viral vector, which includes a circular double-stranded DNA loop into which additional DNA segments can be ligated. As such, in some aspects the poly peptide can be expressed via a plasmid.

Expression vectors can include one or more control or regulatory sequences, selected in some cases on the basis of the host cells to be used for expression, and operably linked to the polynucleotide sequence to be expressed. These regulatory sequences facilitate the expression of the polypeptide, and allow control over various parameters of expression. Non-limiting examples of such control/regulatory sequences can include promoters, enhancers and other expression control elements, such as, for example, polyadenylation signals. In some aspects, control/regulatory sequences can be tailored to target expression of the polynucleotide in specific types of cells and/or tissues. It will be appreciated by those skilled in the art that the design of the expression vector can depend on such factors as the cell type being targeted by the vector, the condition being treated, the desired level of expression of the polypeptide, and the like.

In another aspect, the expression vectors can include viral vectors. Non-limiting examples of viral vectors include retroviral vectors, lentiviral vectors, adenoviral vectors, adeno-associated viral (AAV) vectors, herpes viral vectors, alphavirus vectors, and the like.

The present disclosure additionally provides a variety of pharmaceutical compositions. Such compositions can vary in formulation depending on the mode of delivery, the condition being treated, and the location of the affected cells/tissues. In one aspect, a pharmaceutical composition for treating a condition resulting from abnormally VEGF signaling through membrane-bound VEGF receptors is provided. Such a composition can include at least one of 1) an effective amount of a polypeptide having a sequence region with at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof, or 2) an effective amount of a polynucleotide encoding a polypeptide having a sequence region with at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof and having a polynucleotide sequence that has at least 85% sequence identity to SEQ ID 012, and a pharmaceutically acceptable carrier. In one specific aspect, the composition can be formulated as an ocular pharmaceutical composition.

In some aspects of the invention, a polypeptide or polynucleotide as recited herein or other agent capable of affecting expression of Raver2 can be formulated into a composition for administration by combination with a carrier. A wide range of possible carriers may be selected and used depending on the route of administration and location of the subject to which the composition is to be delivered. For example, water, including deionized water, saline, buffers, isotonic solutions, or other liquid carriers may be used to prepare injectable or parenteral compositions or dosage forms. Additionally, polymers, sugars, celluloses, gelatins, oils, etc. may be used as carriers for formation of an oral composition, and dosage form such as a tablet or capsule. In further aspects, gels, liquids, buffers, polymers, ionic and non-ionic, as well as other molecules may be used as carriers in forming iontophoretic or other transdermal/trans scleral compositions and dosage forms. In addition, selective carrier molecules can be used in order to achieve targeted delivery of Raver2 expression affecting agents, such as those recited herein, to specific cells within a subject.

In another aspect, a method of treating a condition in an individual resulting from abnormally low VEGF signaling through membrane-bound receptors can include decreasing expression of Raver2 in affected cells of the individual to decrease production of soluble VEGF receptor. In another aspect, decreasing expression of Raver2 increases production of membrane-bound VEGF receptors. In one specific example, the soluble VEGF receptor is sFlt-1 and the membrane-bound VEGF receptor is mFlt-1. A variety of conditions are contemplated to be treated, and any such condition is included in the present scope. Non-limiting examples can include pre-eclampsia, heart disease, wound healing, stroke, and the like, including combinations thereof. The present scope additionally includes the prevention of any condition for which a decrease of Raver2 can be used as a treatment. For example, an individual susceptible to pre-eclampsia can be treated to reduce Raver2 during pregnancy to prevent or otherwise minimize the condition.

It is noted that any technique for decreasing Raver2 in an individual is included within the present scope. Non-limiting examples can include siRNAs, antibodies, small molecule antagonists, and the like, including combinations thereof.

TABLE 1
Primers and Oligonucleotides used in study.

| 0ligonucleotide | SEQ ID NO | Sequence |
|---|---|---|
| FLT1-1 | SEQ ID 013 | CTGCAAGATTCAGG |
| | | CACCTA |
| FLT1-2 | SEQ ID 014 | CCTTTTTGTTGCAG |
| | | TGCTCA |
| FLT1-3 | SEQ ID 015 | AAGAAATCACCTAC |
| | | GTGCCGG |
| FLT1-4 | SEQ ID 016 | AGGTTAACCACGTT |
| | | CAGATGG |
| FLT1-5 | SEQ ID 017 | CTGCAAGATTCAGG |
| | | CACCTA |
| FLT1-6 | SEQ ID 018 | AAGTTGACGAGTAA |
| | | TCACAGCTC |
| FLT1-7 | SEQ ID 019 | TAAAGTGGTGGAAC |
| | | TGCTGATG |
| SEA1-1 | SEQ ID 020 | CCACTGCCTACCCT |
| | | CTCACT |
| SEA1-2 | SEQ ID 021 | CCGCTGGGCTCAGT |
| | | GTAGTA |
| HDG4-1 | SEQ ID 022 | CCCACTGAGAGGAC |
| | | AGAGAGA |
| HDG4-2 | SEQ ID 023 | GGCCAGGGTAAAAG |
| | | AGACGA |
| GAPDH-1 | SEQ ID 024 | CATGTTCGTCATGG |
| | | GTGTGAACCA |
| GAPDH-2 | SEQ ID 025 | AGTGATGGCATGGA |
| | | CTGTGGTCAT |
| mouse_Raver1-1 | SEQ ID 026 | ATTTGGCAAGTGTG |
| | | CTACCC |
| mouse_Raver2-2 | SEQ ID 027 | TCGATGGATGGAGA |
| | | ATAGGC |
| mouse_FLT1-1 | SEQ ID 028 | AATGGCCACCACTC |
| | | AAGATT |
| mouse_FLT1-2 | SEQ ID 029 | TTGGAGATCCGAGA |
| | | GAAAATG |
| mouse_FLT1-3 | SEQ ID 030 | ATGAAGTTCCCCTG |
| | | GATGA |
| mouse_FLT1-4 | SEQ ID 031 | ATGCAGAGGCTTGA |
| | | ACGACT |
| mouse_GAPDH-1 | SEQ ID 032 | AACTTTGGCATTGT |
| | | GGAAGGGCTC |
| mouse_GAPDH-2 | SEQ ID 033 | ACCAGTGGATGCAG |
| | | GGATGATGTT |
| iRaver2-1 | SEQ ID 034 | CAGGATGAAGGTAG |
| | | TTACGTT |
| iRaver2-2 | SEQ ID 035 | TTCCAACTCAAACA |
| | | ACGATAA |
| mouse_iRaver2-1A | SEQ ID 036 | GATCCTAAGAAACA |
| | | CCACTGGTCGTTCA |
| | | AGAGACGACCAGTG |
| | | GTGTTTCTTATTA |
| mouse_iRaver2-1B | SEQ ID 037 | AGCTTAATAAGAAA |
| | | CACCACTGGTCGTC |
| | | TCTTGAACGACCAG |
| | | TGGTGTTTCTTAG |
| mouse_iRaver2-2A | SEQ ID 038 | AGATCCTACAAGGG |
| | | TTAGCAGAATATTC |
| | | AAGAGATATTCTGC |
| | | TAACCCTTGTATTA |
| mouse_iRaver2-2B | SEQ ID 039 | AGCTTAATACAAGG |
| | | GTTAGCAGAATATC |
| | | TCTTGAATATTCTG |
| | | CTAACCCTTGTAG |

Human Raver2 (QT00044478) and 18S rRNA (QT00199367) primers were purchased from Qiagen. FLT1-2 primer is sFlt1-specific. Mouse iRaver2 sequences show primers used to generate shRNA plasmids.

### Materials and Methods

### Animals

Male and female C57BL/6J (stock no. 000664), MRL/MpJ (stock no, 000486), and B6EiC3Sn a/APax6^{Sey-Dey}/J(Pax6+/−, stock no. 000391) mice purchased from The Jackson Laboratory (Bar Harbor, Me.) were used. Experimental groups were age and sex matched. All the mice were handled in accordance with the Association for Research in Vision and Ophthalmology (ARVO) Statement for the Use of Animals in Ophthalmic and Vision Research. Experiments were approved by the Institutional Animal Care and Use Committees (IACUCs) of the University of Utah.

### Corneal Microarray and Data Analysis

Corneas were harvested immediately after euthanizing 8-week old female C57BL/6, MRL/MpJ, and Pax6+/− mice and transferred immediately into RNAlater Stabilization Agent (Qiagen, Valencia, Calif., USA). Corneas were then trimmed of any remaining limbus or iris and total RNA was extracted with RNeasy Micro Kit (Qiagen) according to manufacturer's instructions, then submitted to University of Utah Microarray Core Facility where 50 ng of total RNA was used as template for cDNA synthesis for each sample. The polyadenylated fraction of total RNA was primed with oligo dT/T7 RNA polymerase promoter oligonucleotide sequences and cDNA synthesis was accomplished through addition of MMLVRT. Following cDNA synthesis, T7 RNA polymerase and dye-labeled nucleotides are combined with the reaction mixture to simultaneously amplify cRNA and incorporate either cyanin 3-CTP (Cy3) or cyanine 5-CTP (Cy5). Fluorescently labeled cRNA molecules were purified from the reaction mixture using RNeasy mini kit (Qiagen). Sample concentration was determined using a NanaDrop ND-1000 spectrophotometer (Thermo Scientific, Waltham, Mass.). 825 ng of Cy3 and 825 ng of Cy5 labeled cRNA were fragmented and combined with Agilent Hi-RPM Hybridization Buffer. Microarray hybridizations were performed using Agilent SureHyb Hybridization chambers, which were loaded onto a rotisserie in an Agilent Hybrdization oven and incubated at 65° C. for hours with a rotational speed of 10 rpm. Following incubation, the microarray slides were washed for one minute each in Gene Expression Wash Buffer 1 (6× SSPE, 0.005% N-lauroylsarcosine) at RT, then at 31° C. Slides were briefly dipped in a solution of acetonitrile and dried, then scanned in an Agilent G2505B Microarray Scanner. TIF files generated from the scan were loaded into Agilent Feature Extraction.

Software version 10.1.1.1, which automatically positions a grid and finds the centroid position of each feature on the array, calculates feature intensities and background, then records data as a tab-delimited text file.

Each Cy3 or Cy5 hybridization was treated as an individual biological replicate in subsequent data analysis. Microarray intensity data was filtered to remove control features and any features flagged as non-uniform or feature population outliers. Any remaining values for each microarray probe were averaged to yield a single value for each probe sequence for each sample. Values were log2-transformed and quantile normalized. Normalized data was uploaded to GeneSifter (www.geospiza.com) for differential expression analysis. Differentially expressed genes were selected using ANOVA, requiring at least 2-fold differential expression and a Benjamini and Hochberg-corrected p value <0.05. Log 2 intensity data from all samples and all genes was clustered in R using Ward's method and Euclidean distance. Heatmaps were generated in R using the heatmap.2 function in the gplots library from BioConductor. Genes correlated or anti-correlated with Raver2 expression were clustered by first calculating the mean expression value for each gene, and then calculating the deviation from the mean for each gene in each sample. These deviations were hierarchically clustered using Euclidean distance and complete linkage. The color scale represents deviation from mean expression for each gene, with increased expression displayed in red, and decreased expression in green.

Cell Culture, siRNA, AMO, and Plasmid Transfection, and Total RNA Preparation

HUVECs (Lonza, Walkersville, Md., USA) were cultured in endothelial basal medium (EBM) supplemented with Single Quot Kit and growth factors according to the manufacturer's instructions. To prevent loss of endothelial cell properties, cultures were limited to passages four through seven. siRNAs targeting Raver2 and non-specific control siRNA were purchased as predesigned FlexiTube siRNAs (Qiagen). Sequences of the Raver2-specific siRNAs are given in Table 1. For siRNA transfection, 2×10⁵ cells/well (6-well plate) HUVECs were transfected with 30 pmol siRNA using lipofectamine RNA iMax (Life Technologies, Grand Island, N.Y., USA) according to the manufacturer's protocol. For sequential siRNA/AMO treatments, transfection with either U1 or standard AMO was performed as described previously (37), 48 hours following siRNA transfection. For plasmid transfection, 1×10⁶ cells underwent electroporation with 2 μg pCMV Raver2-FLAG (OriGene, Rockville, Md., USA) or empty pCMV vector using Nucleofector (Lonza) according to manufacturer's protocol. Total RNA was isolated 48 hours after transfection using RNeasy mini kit (Qiagen) with DNaseI treatment according to manufacturer's protocol.

### HUVEC Immunofluorescence Staining

HUVECs were fixed with 4% paraformaldehyde in PBS for 20 minutes at RT, followed by two PBS washes. Cells were permeabilized with methanol, followed by an additional three PBS washes and incubation in blocking buffer (5% donkey serum, 0.02% tritonX-100 in PBS) for 30 minutes at RT. Cells were then incubated with 1:100 PTB antibody (32-4800, Life Technologies) and 1: 100 Raver2 antibody (sc-165338, Santa Cruz Biotechnology, Santa Cruz, Calif., USA) in blocking buffer for 1 hour at RT followed by four PBS washes. Cells were then incubated in secondary antibody (1:1000) in blocking buffer for 30 min at RT followed by four PBS washes. Nuclear staining was performed with DAPI, samples were mounted with Fluorogel (Electron Microscopy Sciences, USA), and images captured using an Olympus Confocal Microscope (FV1000).

cDNA Synthesis and Quantitative RT-PCR

cDNAs were synthesized from total RNA (corneal or HUVEC) using the Omniscript RT kit (Qiagen) with oligo-dT (dT20) primers according to the manufacturer's protocol. Real-time PCR used the QuantiTect SYBR Green PCR Kit (Qiagen) with amplification performed on a GeneAmp 5700 Thermocycler (ABI, Foster City, Calif.). Wild-type HUVEC cDNA was diluted serially to construct a fivepoint standard curve, which was run in parallel on the same plate for each experiment. Expression levels were normalized to internal control gene GAPDH. For three-primer PCR, cDNA was synthesized using random hexamers and RI-PCR was carried out using primers FLT -5, FLT1-6, and FLT1-7.

(Table 1) followed by ImageJ analysis (U.S. National Institutes of Health, Bethesda, Md., USA).

### Immunoprecipitation and Western Blotting

Cells were lysed RIPA buffer (50mM Tris, pH 8, 150 mM NaCl, 1% NP40, 0.1% SDS, 0.5% sodium deoxycholate, protease inhibitors ). 5 μg of Raver2 (Santa Cruz) or PTB (Life Technologies) antibody was added to 300 μg of cell lysate and incubated for six hours at 4° C. in spin wheel. 30 μl of protein agarose A/G (Santa Cruz) was centrifuged at 1000 g for one minute and the supernatant was aspirated. Beads were washed three times in 50 μL IP buffer (Dynabeads Co-Immunoprecipitation Kit, Life Technologies), and equilibrated beads were added to the lysate/antibody homogenate and incubated overnight at 4° C. in a spin wheel. Beads were collected by centrifugation, washed in PBS (7 times) and eluted by heating to 95° C. for two minutes in Laemmli Buffer (BioRad, Hercules, Calif., USA). Samples were run on a 10% SDSPAGE gel, and Western blotting was performed using standard techniques with Raver2 (Santa Cruz) and PTB (Life Technologies) antibodies. The same protocol was used for cell lysate preparation and Western blotting analysis of Raver2 following siRNA or plasmid transfection in HUVECs using Raver2 (Santa Cruz), FLAG (TA50011, OriGene), and GAPDH (ab9485, AbCam, Cambridge, Mass., USA) antibodies.

### Enzyme-Linked Immunosorbent Assay (ELISA)

Cell culture supernatant was harvested 72 hrs following transfection of HUVEC with siRNA or plasmid, and ELISA was performed for sFlt1 using the Quantikine Kit (R&D Systems, Inc, Minneapolis, Minn.) according to the manufacturer's instructions.

### RNA Immunoprecipitation (RIP)

RIP assays were carried out using the Magna RIP Kit (Millipore) according to manufacturer's protocol. Cells were harvested by scraping in ice-cold PBS and collected by centrifugation at 3000 rpm for 5 minutes at 4° C. The cells were subsequently lysed and cell extracts were made with RIP Lysis Buffer (Magna RIP Kit, Millipore). The lysates (100 μg protein per sample) were incubated with 5 μg antibody (Raver2, Santa Cruz; or PTB, Life Technologies) with magnetic A/G beads at 4° C. overnight with gentle rotation. IgG1 isotype antibody (02-6100, Life Technologies) was used as control. Beads were pelleted with a magnetic separator, washed three times with wash buffer (Magna RIP Kit, Millipore), and treated with proteinase K followed by RNA extraction with phenol-chloroform. cDNA was synthesized from 50 ng purified total RNA (DNaseI treated using random hexamers and Sensiscript RT Kit (Qiagen) according to manufacturer's protocol. Reactions with or without reverse transcriptase were performed for each sample, and resulting cDNAs were analyzed by RT-PCR using Taq DNA Polymerase (NEB, Ipswich, Mass.), or qRT-PCR as described above.

### Corneal Injections and Imaging

shRNA expression cassettes were created based upon iRaver2-1 and iRaver2-2 siRNA sequences. Complementary oligonucleotides were constructed and cloned into pSilencer4.1 CMV Neo vector and verified by sequencing. shRNA-bearing plasmids were injected into the corneas of anesthetized C57BL/6 mice (8 weeks of age) under direct microscopic observation. A nick was made through the epithelium into the anterior corneal stroma with a 0.5 inch, 30-gauge needle on a 10 μL gas-tight syringe (Hamilton, Reno, Nev.) and 4μL of 1 μg/μL solution was gently injected into the stroma to deliver the plasmid.

Raver2-FLAG or empty vector plasmic's (or buffer only control) were similarly delivered via subconjunctival injection (10 μL volume of 1 μg/μL solution per injection) at the corneal limbus of Pax6+/− eyes (5 weeks of age). In vivo images were captured by CCD camera (Nikon) under a dissecting microscope. CD31 staining and cornea flat mount preparation was carried out and masked analysis performed as previously described using ImageJ.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A method of treating a condition resulting from abnormally high VEGF signaling through membrane-bound VEGF receptors, comprising:
administering to a subject in need of such treatment an effective amount of a polypeptide having a sequence region with at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof, wherein the polypeptide upregulates soluble VEGF receptor production in affected cells to decrease the abnormally high VEGF signaling through the membrane-bound VEGF receptors.

2. The method of claim 1, wherein the sequence region has 100% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof.

3. The method of claim 1, wherein the polypeptide is Raver2.

4. The method of claim 1, wherein the polypeptide has at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, or SEQ ID 011.

5. The method of claim 1, wherein the polypeptide has 100% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, or SEQ ID 011.

6. The method of claim 1, wherein administering the effective amount of the polypeptide further includes administering an effective amount of a polynucleotide encoding the polypeptide or polypeptide region, wherein the polynucleotide has at least 85% sequence identity to SEQ ID 012.

7. The method of claim 6, wherein the polynucleotide has at least 90% sequence identity to SEQ ID 012.

8. The method of claim 6, wherein the polynucleotide has at least 95% sequence identity to SEQ ID 012.

9. The method of claim wherein the polynucleotide has 100% sequence identity to SEQ ID 012.

10. The method of claim 1, wherein the condition is selected from the group consisting of cancer, macular degeneration, diabetic retinopathy, rheumatoid arthritis, corneal injury, corneal transplant rejection, or a combination thereof.

11. The method of claim 11, wherein the condition is selected from the group consisting of macular degeneration, diabetic retinopathy, conical injury, conical transplant rejection, or a combination thereof.

12. A method of treating a condition in a subject resulting from abnormally high VEGF signaling through membrane-bound receptors, comprising:
increasing expression of Raver2 in affected cells of the subject to increase production of soluble VEGF receptors.

13. The method of claim 12, wherein increasing expression of Raver2 decreases production of membrane-bound VEGF receptors.

14. The method of claim 12, wherein the condition is selected from the group consisting of cancer, macular degeneration, diabetic retinopathy, rheumatoid arthritis, corneal injury, corneal transplant rejection, or combinations thereof.

15. The method of claim 12, wherein the condition is selected from the group consisting of macular degeneration, diabetic retinopathy, conical injury, corneal transplant rejection, or combinations thereof.

16. A method of treating a condition in a subject resulting from abnormally low VEGF signaling through membrane-bound receptors, comprising:
decreasing expression of Raver2 in affected cells of the subject to decrease production of soluble VEGF.

17. The method of claim 17, wherein decreasing expression of Raver2 increases production of membrane-bound VEGF receptors.

18. The method of claim 17, wherein t0he condition is selected from the group consisting of
preeclampsia, heart disease, wound healing, stroke, or a combination thereof.

19. A pharmaceutical composition for treating a condition resulting from abnormally high VEGF signaling through membrane-bound VEGF receptors, comprising:
at least one of 1) an effective amount of a poly peptide having a sequence region with at least 95% sequence identity to at least one of SEQ 001, SEQ ID 002, SEQ lD 003, SEQ ID 004, SEQ ID 005, SEQ ID 006, SEQ ID 007, SEQ ID 008, SEQ ID 009, SEQ ID 010, SEQ ID 011, or a combination thereof, or 2) an effective amount of a polynucleotide encoding a polypeptide having a sequence region with at least 95% sequence identity to at least one of SEQ ID 001, SEQ ID 002, SEQ ID 003, SEQ ID 004, SEQ ID 005, SEQ ID 006, SEQ ID 007, SEQ ID 008, SEQ ID 009, SEQ ID 010, SEQ ID 011, or a combination thereof and having a polynucleotide sequence that has at least 85% sequence identity to SEQ ID 012; and
a pharmaceutically acceptable carrier.

20. The composition of claim 1, formulated as an ocular pharmaceutical composition.


SEQUENCE LISTING (39 sequences)

SEQ ID NO: 1 - protein, 691 aa, Homo sapiens
MAAAAGDGGGEGGAGLGSAAGLGPGPGLRGQGPSAEAHEGAPDPMPAALHPEEVAARLQRMQRELSNRRKILVKNLPQDSNCQEVHDLLKDYDLKYCYVDRNKRTAFVTLLNGEQAQNAIQMFHQYSFRGKDLIVQLQPTDALLCITNVPISFTSEEFEELVRAYGNIERCFLVYSEVTGHSKGYGFVEYMKKDFAAKARLELLGRQLGASALFAQWMDVNLLASELIHSKCLCIDKLPSDYRDSEELLQIFSSVHKPVFCQLAQDEGSYVGGFAVVEYSTAEQAEEVQQAADGMTIKGSKVQVSFCAPGAPGRSTLAALIAAQRVMHSNQKGLLPEPNPVQIMKSLNNPAMLQVLLQPQLCGRAVKPAVLGTPHSLPHLMNPSISPAFLHLNKAHQSSVMGNTSNLFLQNLSHIPLAQQQLMKFENIHTNNKPGLLGEPPAVVLQTALGIGSVLPLKKELGHHHGEAHKTSSLIPTQTTITAGMGMLPFFPNQHIAGQAGPGHSNTQEKQPATVGMAEGNFSGSQPYLQSFPNLAAGSLLVGHHKQQQSQPKGTEISSGAASKNQTSLLGEPPKEIRLSKNPYLNLASVLPSVCLSSPASKTTLHKTGIASSILDAISQGSESQHALEKCIAYSPPFGDYAQVSSLRNEKRGSSYLISAPEGGSVECVDQHSQGTGAYYMETYLKKKRVY

SEQ ID NO: 2 - protein, 659 aa, Homo sapiens
PSAEAHEGAPDPMPAALHPEEVAARLQRMQRELSNRRKILVKNLPQDSNCQEVHDLLKDYDLKYCYVDRNKRTAFVTLLNGEQAQNAIQMFHQYSFRGKDLIVQLQPTDALLCITNVPISFTSEEFEELVRAYGNIERCFLVYSEVTGHSKGYGFVEYMKKDFAAKARLELLGRQLGASALFAQWMDVNLLASELIHSKCLCIDKLPSDYRDSEELLQIFSSVHKPVFCQLAQDEGSYVGGFAVVEYSTAEQAEEVQQAADGMTIKGSKVQVSFCAPGAPGRSTLAALIAAQRVMHSNQKGLLPEPNPVQIMKSLNNPAMLQVLLQPQLCGRAVKPAVLGTPHSLPHLMNPSISPAFLHLNKAHQSSVMGNTSNLFLQNLSHIPLAQQQLMKFENIHTNNKPGLLGEPPAVVLQTALGIGSVLPLKKELGHHHGEAHKTSSLIPTQTTITAGMGMLPFFPNQHIAGQAGPGHSNTQEKQPATVGMAEGNFSGSQPYLQSFPNLAAGSLLVGHHKQQQSQPKGTEISSGAASKNQTSLLGEPPKEIRLSKNPYLNLASVLPSVCLSSPASKTTLHKTGIASSILDAISQGSESQHALEKCIAYSPPFGDYAQVSSLRNEKRGSSYLISAPEGGSVECVDQHSQGTGAYYMETYLKKKRVY

SEQ ID NO: 3 - protein, 72 aa, Homo sapiens
RKILVKNLPQDSNCQEVHDLLKDYDLKYCYVDRNKRTAFVTLLNGEQAQNAIQMFHQYSFRGKDLIVQLQPT

SEQ ID NO: 4 - protein, 79 aa, Homo sapiens
ALLCITNVPISFTSEEFEELVRAYGNIERCFLVYSEVTGHSKGYGFVEYMKKDFAAKARLELLGRQLGASALFAQWMDV

SEQ ID NO: 5 - protein, 79 aa, Homo sapiens
KCLCIDKLPSDYRDSEELLQIFSSVHKPVFCQLAQDEGSYVGGFAVVEYSTAEQAEEVQQAADGMTIKGSKVQVSFCAP

SEQ ID NO: 6 - protein, 678 aa, Homo sapiens
MAAAAGDGGGEGGAGLGSAAGLGPGPGLRGQGPSAEAHEGAPDPMPAALHPEEVAARLQRMQRELSNRRKILVKNLPQDSNCQEVHDLLKDYDLKYCYVDRNKRTAFVTLLNGEQAQNAIQMFHQYSFRGKDLIVQLQPTDALLCITNVPISFTSEEFEELVRAYGNIERCFLVYSEVTGHSKGYGFVEYMKKDFAAKARLELLGRQLGASALFAQWMDVNLLASELIHSKCLCIDKLPSDYRDSEELLQIFSSVHKPVFCQLAQDEGSYVGGFAVVEYSTAEQAEEVQQAADGMTIKGSKVQVSFCAPGAPGRSTLAALIAAQRVMHSNQKGLLPEPNPVQIMKSLNNPAMLQVLLQPQLCGRAVKPAVLGTPHSLPHLMNPSISPAFLHLNKAHQNLSHIPLAQQQLMKFENIHTNNKPGLLGEPPAVVLQTALGIGSVLPLKKELGHHHGEAHKTSSLIPTQTTITAGMGMLPFFPNQHIAGQAGPGHSNTQEKQPATVGMAEGNFSGSQPYLQSFPNLAAGSLLVGHHKQQQSQPKGTEISSGAASKNQTSLLGEPPKEIRLSKNPYLNLASVLPSVCLSSPASKTTLHKTGIASSILDAISQGSESQHALEKCIAYSPPFGDYAQVSSLRNEKRGSSYLISAPEGGSVECVDQHSQGTGAYYMETYLKKKRVY

SEQ ID NO: 7 - protein, 20 aa, Homo sapiens
IHTNNKPGLLGEPPAVVLQT

SEQ ID NO: 8 - protein, 20 aa, Homo sapiens
AHTNNKPGLLGEPPAMVLQP

SEQ ID NO: 9 - protein, 20 aa, Homo sapiens
AASKNQTSLLGEPPKEIRLS

SEQ ID NO: 10 - protein, 20 aa, Homo sapiens
VASKNQTSLLGEPPKEIRLS

SEQ ID NO: 11 - protein, 514 aa, Homo sapiens
VTGHSKGYGFVEYMKKDFAAKARLELLGRQLGASALFAQWMDVNLLASELIHSKCLCIDKLPSDYRDSEELLQIFSSVHKPVFCQLAQDEGSYVGGFAVVEYSTAEQAEEVQQAADGMTIKGSKVQVSFCAPGAPGRSTLAALIAAQRVMHSNQKGLLPEPNPVQIMKSLNNPAMLQVLLQPQLCGRAVKPAVLGTPHSLPHLMNPSISPAFLHLNKAHQSSVMGNTSNLFLQNLSHIPLAQQQLMKFENIHTNNKPGLLGEPPAVVLQTALGIGSVLPLKKELGHHHGEAHKTSSLIPTQTTITAGMGMLPFFPNQHIAGQAGPGHSNTQEKQPATVGMAEGNFSGSQPYLQSFPNLAAGSLLVGHHKQQQSQPKGTEISSGAASKNQTSLLGEPPKEIRLSKNPYLNLASVLPSVCLSSPASKTTLHKTGIASSILDAISQGSESQHALEKCIAYSPPFGDYAQVSSLRNEKRGSSYLISAPEGGSVECVDQHSQGTGAYYMETYLKKKRVY

SEQ ID NO: 12 - DNA, 2440 bp, Homo sapiens
gttactggccattccaaaggctatggatttgtggaatacatgaaaaaggactttgctgcaaaggctagactggagctattgggtagacagttgggagcatcagcactctttgcacaatggatggatgttaatctattggcttcagagctcattcattctaagtgcctttgtattgataaactccccagtgactacagggattcagaagagctgttgcaaattttttccagtgtccataaacctgtgttttgccagcttgcacaggatgaaggtagttacgttggtggctttgcagtggttgaatatagcactgcggagcaggctgaagaggtccagcaggcagcagacggtatgaccatcaagggcagcaaagtccaggtttccttctgtgctcctggagcgccagggcgaagtacattagcagcattgatagcggctcaacgtgtgatgcacagtaatcaaaagggcttacttccagagccaaatccagtacaaattatgaaaagtttaaacaaccctgccatgttgcaagttcttctacagccccagttatgtggacgagctgttaaaccagccgttcttggaacacctcacagcttgccacatctgatgaatccatccatctctcctgcatttttacatttgaataaagcacatcagagctcagttatgggtaatacttctaatttattccttcagaatctttctcatataccactggcacaacaacaattaatgaagtttgagaatattcatactaataataaacccggcttacttggagagcccccagctgtggtacttcagactgcactagggatagggtcagtgcttccattgaaaaaggagttgggacatcatcatggagaagcacataaaacatctagcctgattccaactcaaacaacgataacagctggaatgggcatgttaccattctttccaaatcagcacattgctggacaggctgggccagggcacagtaatactcaggagaaacagccagccacggtgggaatggcagaaggaaatttctcagggtcgcagccttatcttcagagctttccaaaccttgctgctggaagcttgctggtgggacaccataagcagcagcaaagccagccaaaaggcacagagataagttcaggggctgcctctaagaatcaaacttcactcttgggagaaccaccaaaagaaattcggctcagtaaaaatccatacttgaatttggcaagtgtgttgcccagtgtgtgcttatcatcccctgcaagtaaaaccactcttcataagactggaattgcaagcagcattctggatgcaatctctcagggaagtgaatcacaacacgcattggaaaagtgcattgcttattctccaccttttggtgattatgcacaggtgtcatctttaagaaatgaaaagcgaggctcatcatatctcatctctgccccagaaggtggttcagtggaatgtgttgaccagcattctcagggcacaggagcatattacatggaaacttacttaaaaaagaagcgagtatactgagttaagctctctcctaataaccccctaaggttctctgcagtatctctgctgttcatcgctgccttaacttcattcaaactagccatatcctttaaatacgggtttatagtttcccagaaggaactgtgttgtgcagcaggcagaaatgccaaataaaaaatagcaataagaagagacatcaattgaggacattttccactagagtcagatgcatcttaatcagtgtgctaacttacaatccattggagaaactaacaatgttccaatattccttgatttaggactgggaataattctgacaccatatataaaagaagtttgaagcaataaatgggaaacagtgttgtttcctgagccttagaagaatgagtttcttataagccaatgtgcatttcaatttactttccctagaagtaattgcatttatagatttttaggacttttcctagaactacaagggtgcaagtaccaaatgtgctgatggtaatattataaagatgctctacatgcacactcaattacgcccacaccagccatgcaaaattaaacccggtaagagagtgacagtgatatccaaggtatccactactaccgatgctttaagatgttgtttaccatttataccttctgtgaaaaatagaaggtattttaagaaaagtaactttggtgatctaataaaagggtttggcaaactacaccctgggtccacatttggtctaccacccgcttttgtgctgcctgcaagctaagactaatgtttacatttttaaatggttggaggaaaaaagaaaacttatgacatgaaatttatatgaaattcagatttcagtgtccataaataaacttttattgggaaaaaaaaaaaaaaaaaaaaaaaaaaaaaaaaaaaaaaaaa

SEQ ID NO: 13 - DNA, 20 bp, Homo sapiens
ctgcaagattcaggcaccta

SEQ ID NO: 14 - DNA, 20 bp, Homo sapiens
cctttttgttgcagtgctca

SEQ ID NO: 15 - DNA, 21 bp, Homo sapiens
aagaaatcacctacgtgccgg

SEQ ID NO: 16 - DNA, 21 bp, Homo sapiens
aggttaaccacgttcagatgg

SEQ ID NO: 17 - DNA, 20 bp, Homo sapiens
ctgcaagattcaggcaccta

SEQ ID NO: 18 - DNA, 23 bp, Homo sapiens
aagttgacgagtaatcacagctc

SEQ ID NO: 19 - DNA, 22 bp, Homo sapiens
taaagtggtggaactgctgatg

SEQ ID NO: 20 - DNA, 20 bp, Homo sapiens
ccactgcctaccctctcact

SEQ ID NO: 21 - DNA, 20 bp, Homo sapiens
ccgctgggctcagtgtagta

SEQ ID NO: 22 - DNA, 21 bp, Homo sapiens
cccactgagaggacagagaga

SEQ ID NO: 23 - DNA, 20 bp, Homo sapiens
ggccagggtaaaagagacga

SEQ ID NO: 24 - DNA, 24 bp, Homo sapiens
catgttcgtcatgggtgtgaacca

SEQ ID NO: 25 - DNA, 24 bp, Homo sapiens
agtgatggcatggactgtggtcat

SEQ ID NO: 26 - DNA, 20 bp, Mouse
atttggcaagtgtgctaccc

SEQ ID NO: 27 - DNA, 20 bp, Mouse
tcgatggatggagaataggc

SEQ ID NO: 28 - DNA, 20 bp, Mouse
aatggccaccactcaagatt

SEQ ID NO: 29 - DNA, 21 bp, Mouse
ttggagatccgagagaaaatg

SEQ ID NO: 30 - DNA, 19 bp, Mouse
atgaagttcccctggatga

SEQ ID NO: 31 - DNA, 20 bp, Mouse
atgcagaggcttgaacgact

SEQ ID NO: 32 - DNA, 24 bp, Mouse
aactttggcattgtggaagggctc

SEQ ID NO: 33 - DNA, 24 bp, Mouse
accagtggatgcagggatgatgtt

SEQ ID NO: 34 - DNA, 21 bp, Homo sapiens
caggatgaaggtagttacgtt

SEQ ID NO: 35 - DNA, 21 bp, Homo sapiens
ttccaactcaaacaacgataa

SEQ ID NO: 36 - DNA, 55 bp, Mouse
gatcctaagaaacaccactggtcgttcaagagacgaccagtggtgtttcttatta

SEQ ID NO: 37 - DNA, 55 bp, Mouse
agcttaataagaaacaccactggtcgtctcttgaacgaccagtggtgtttcttag

SEQ ID NO: 38 - DNA, 56 bp, Mouse
agatcctacaagggttagcagaatattcaagagatattctgctaacccttgtatta

SEQ ID NO: 39 - DNA, 55 bp, Mouse
agcttaatacaagggttagcagaatatctcttgaatattctgctaacccttgtag